(19) United States
(12) Patent Application Publication
(10) Pub. No.: US 2012/0220489 A1
(43) Pub. Date: Aug. 30, 2012

(54) CALIBRATION REAGENT AND USES THEREOF

(76) Inventors: Maziar Assadi Gehr, Basel (CH); Everson Nogoceke, Basel (CH)

(21) Appl. No.: 13/509,984

(22) PCT Filed: Nov. 12, 2010
(86) PCT No.: PCT/EP10/67379

§ 371 Date: May 15, 2012

(30) Foreign Application Priority Data
Nov. 16, 2009 (EP) 09176130.4

(51) Int. Cl. C40B 30/04; C07K 19/00

(52) U.S. Cl. 506/9; 530/350; 530/363

(57) ABSTRACT

The present invention provides a calibration reagent comprising a peptide conjugated to a protein carrier via a linker, wherein said peptide comprises an epitope of interest and the use thereof.

Reverse phase protein arrays (RPA) have been developed and established in the recent years as a convenient method to analyze focused sets of proteins representing key analytes of different signal transduction cascades in minute amounts of biological samples (e.g. cell lysates, tissue lysates, or body fluids). Relative differences of protein expression, representing not only the abundance of specific key proteins, but also activated, post-translationally modified (e.g. phosphorylated) forms of such key proteins can describe and classify e.g. specific treatment effects of pharmaceutical compounds given to cell cultures e.g. inhibitory effects of drug candidates on kinases, or describe and classify different disease states e.g. sub-types of tumors in their different progression states. RPA can perform comparative measurements of many samples in parallel, e.g. samples from differently treated cell cultures or samples from different disease populations. Significant changes of protein expression or protein activation patterns to be found in distinct sample cohorts will foster e.g. the identification of most efficient drug candidates, the elucidation of treatment induced mode-of-action schemes or the discovery of new diagnostic/prognostic disease markers.

Immunoaffinity assays such as used in Reverse Phase Protein Arrays (RPA) are based on specific interactions between an affinity reagent and a protein of interest. The assay comprises the immobilization of the biological samples on the array forming the sample spots. The sampled array is incubated with an affinity reagent, i.e. an antibody, and the subsequently formed complex of affinity reagent and protein of interest is measured by the generated detection signal e.g. a luminescence signal. Each array is stained with an analyte-specific affinity reagent, which can be labeled or is incubated with a secondary detection reagent. Formed complexes are detected by various means (colorimetric, fluorescence, chemiluminescence etc.). Typically RPA measure relative changes of expression or activation signals between different samples.

The quantitative analysis of samples requires the use of calibration reagents. Currently, for protein analytes, the calibration reagents are recombinant proteins having the same amino sequence as the analyte. For example, patent application WO2007/048436A1 describes calibration curves for Reverse Phase Protein Microarrays whereby different concentrations of purified protein of interest (Akt) were added to spotting buffer comprising BSA or rat serum. However, the production of recombinant protein presenting the correct epitope is time-consuming and often not successful. In particular for phosphorylated epitopes, so far no reliable calibration reagents are available.

Therefore, there is a need for a reagent designed to provide universal applicability with choosable specificity for the different analyte epitopes of interest. This would allow to calibrate results from experiments performed, e.g. at separated times, by different lab personal, on different devices or on arrays constructed in different print runs. Also the linear range of the protein-specific RPA signals to be generated by the respective affinity reagent can be optimally pre-defined.

Therefore, the present invention provides a calibration reagent comprising a peptide which is attached via a linker to a protein carrier, wherein said peptide comprises an epitope of interest. Preferably, said epitope of interest is phosphorylated.

With this calibration reagent reliable standard curves can be generated for quantifying protein with an RPA or another affinity assay. RPAs are constructed by the deposition of small sample volumes e.g. of cell or tissue lysates, onto highly binding substrate surfaces using often a robotic microarrayer. Each lysate spot on the substrate contains the full complement of cellular proteins and analytes. Hundreds of samples can be spotted in parallel into one microarray allowing high throughput cross-comparisons of samples in the same assay. Replicate arrays containing the same set of samples, can be easily produced from the same initial volume of sample material, since consumption of sample volume per spot is extremely low.

The calibration reagent of the current invention is particular useful for quantifying proteins which comprise a phosphorylated epitope of interest.

The term “epitope of interest” refers to a part of a polypeptide which is recognized by the affinity reagent of interest. The affinity reagent of interest is preferably specific for the epitope of interest.

The term “epitope peptide” as used herein refers to the peptide comprising the epitope of interest. The epitope peptide is preferably between 12 and 25 amino acids long. More preferably, the length of the peptide is 12 to 20, most preferably 14 to 17 amino acids long.

The epitope of interest can be modified, e.g. phosphorylated. The term “phosphorylated epitope” as used herein refers to an epitope which comprises at least one amino acid with a phosphate group. Preferably, the epitope of interest comprises 1 to 5 phosphorylated amino acids. Preferably, the position of the modified amino acid is approximately in the middle of the epitope peptide. Fore example in a peptide of 15 amino acids length, the modified amino acid is preferably at position 7, 8 and/or 9 (see FIG. 2C). Methods for modifying an amino acid (e.g. to phosphorylate) are well known to the skilled person in the art.

The epitope peptide is covalently bound to the protein carrier via a linker (see FIG. 1A), whereby the epitope peptide is covalently bound to the linker and the linker is covalently bound to the protein carrier. In a preferred embodiment, the linker is covalently bound to the free N-terminal Cysteine (Cys) group of the BSA, wherein a free Cys group is a cysteine residue which is not involved in a disulfide bridge.

The epitope peptide can be attached to the protein carrier in essentially two steps:

Step 1) The linker is conjugated to the epitope peptide, wherein said linker is preferably labeled with a tag. The linker can be attached to the N- or C-terminus of the peptide. Preferably, the linker is attached to the N-terminus of the peptide.

Step 2) the free end of the linker is conjugated to the protein carrier.

The linker or spacer is a peptide comprising 2 to 10, preferably 2 to 5, more preferably 3 to 4 natural or unnatural amino acids. Natural amino acids are naturally occurring amino acids such as in particular alanine, cysteine, lysine, histidine, arginine, aspartate, glutamate, serine, threonine, methionine, glycine, valine, leucine, isoleucine, asparagine, glutamine, proline, tryptophane, phenylalanine, tyrosine. Unnatural amino acids are amino acids which do not naturally occur. Examples for unnatural amino acids are 8-amino-3, 6 dioxa-octanoic acid (Doa) and aminooxy-acetic acid.

The linker is hydrophilic and can comprise only natural amino acids or only unnatural amino acids or a mixture of both, natural and unnatural amino acids. Preferably, the linker comprises one or more of the following natural amino acids: cysteine, lysine, histidine, arginine, aspartate, glutamate. Also preferably, the linker comprises one or more Doa. More preferably, the linker is Cysteine-Doa-Doa.

Preferably, the linker is labeled with Dabsyl.

Methods to produce peptides with a specific amino acid sequence are well known to the skilled person in the art. A suitable method is e.g. is the solid phase synthesis described in Merrifield, Science 1986, 232:341-347 (method) and Carpino et al., J. Am. Chem. 1990, 112: 9651-52 (reagents).

The protein carrier is a protein which unspecifically binds to surfaces. Preferably, the protein carrier is a protein of at least 20 kDa and shows no or low cross reactivity with the affinity reagent used in an affinity assay. The protein carrier is preferably an albumin, more preferably a serum albumin, such as e.g. bovine serum albumin (BSA) or human serum albumin. The preferred serum albumin is BSA.

An “affinity reagent of interest” is a reagent which recognizes and binds the epitope of interest. Preferably, the affinity reagent of interest is specific and selective for the epitope of interest. The affinity reagent can be an antibody, an aptamer, or a designed ankyrin repeat protein (DARPin). Preferably, the affinity reagent is an antibody.

An “antibody of interest” can be any antibody. Preferably, said antibody is an IgG antibody, more preferably a monoclonal antibody. The antibody of interest includes but is not limited to humanized antibody and rodent antibody. A rodent antibody includes but is not limited to a mouse, rabbit and rat antibody. Preferably, the antibody is a rabbit monoclonal antibody.

An “aptamer of interest” is a single-stranded RNA or DNA oligonucleotide 15 to 60 base in length that bind with high affinity to the epitope of interest.

A “designed ankyrin repeat protein” or “DARPin” is a binding molecule comprising at least one ankyrin repeat. An ankyrin repeat is a motif in proteins consisting of two alpha helices separated by loops, which can be selected to recognize specifically a wide variety of target proteins. The typical length of an ankyrin repeat is 33 amino acids. Unlike antibodies they do not contain any disulfide bonds and are found in all cellular compartments.

Furthermore, the present invention provides the use of the calibration reagent as described above for the generation of a standard curve. The present invention provides a method for generating a standard curve comprising the steps of:

a) immobilizing the above described calibration reagent in two or more concentrations on an array,

b) incubating said array with a detectable affinity reagent of interest,

c) measuring the signal intensity of the bound affinity reagent for each of the two or more concentrations of the calibration reagent, and

d) correlating the signal intensity with amount of epitope of interest.

A standard or calibration curve is a quantitative research tool, a method of plotting assay data that is used to determine the concentration of a substance, i.e. the concentration of the epitope of interest.

The term “bound affinity reagent” refers to the affinity reagent forming a complex with a protein or peptide comprising the epitope of interest. Formed complexes are detected by various means such as for example colorimetric, fluorescence, or chemiluminescence.

An affinity reagent can be detected, by a detectable label attached to the affinity reagent. Preferably, said label is a fluorophore, allowing thereby to determining the amount of bound antibody by the fluorescence intensity. Other suitable labels are e.g. alkaline phosphatase (AP) and horseradish peroxidase (HRP).

An affinity reagent can also be detected by a secondary detection reagent. A secondary detection reagent is a labeled molecule which selectively binds the affinity reagent. The bound affinity reagent on the microarray can be detected for example by using a second antibody or a Fab fragment, which is labeled and recognizes species-specific epitopes of the affinity reagent. Suitable labels include but are not limited to fluorophore, biotin, horseradish peroxidase, and isotopes. Preferably, the secondary detection reagent (e.g. a secondary antibody) is labeled with a fluorophore.

The amount of affinity reagent bound to the calibration reagent is preferably detected by an optical signal such as a fluorescence signal.

The amount of bound affinity reagent of interest is correlated with amount of epitope of interest by measuring the detectable signal of the affinity reagent and attribute each signal a concentration of the epitope of interest. These results are displayed in a standard curve. A standard curve can be drawn by plotting the determined amount of bound affinity reagent of interest for each concentration of the epitope of interest (on the Y axis) versus the concentration of the epitope of interest (on the X axis). The amount of bound affinity reagent is usually displayed as the strength of the detected signal (signal intensity). Preferably said signal is an optical signal, more preferably fluorescence intensity. Typically, for the purpose of generating a standard curve, the spots on the array comprise different concentrations of the calibration reagent, preferably as a serial dilution (e.g. a series of two-fold dilution).

The concentration of the epitope of interest in a known concentration of calibration reagent is obtained by determining the peptide:carrier ratio, which is the number of peptides conjugated to one protein carrier.

Methods for determining the peptide:carrier ratio are well known to the skilled person in the art. A suitable method is e.g. a method comprising the following steps: step 1: determining the conjugated peptide concentration by for example photometric absorbance measurement, whereby the peptides or the linker attached to the peptides are preferably labeled with a tag (e.g. Dabsyl); step 2: determining the total protein concentration of the conjugated product via Bradford test and step 3: calculating the peptide:protein ratio. Preferably, the linker is labeled with tag such as e.g. Dabsyl, which allows to determine the peptide:protein carrier ratio. Suitable ratios for use in the methods of the invention can be up to 10 and higher. Preferably, the ratio is lower than 3, more preferably, the ratio is equal to or lower than 1, most preferably the ratio is between 0.3 and 1.

The affinity reagent of interest is incubated on the array for at least 30 minutes, preferably for more than 1 hour, more preferably for 1 to 16 hours, most preferably about 12 hours (12 hours ±30 minutes). The excess of affinity reagent is removed and preferably the array is washed before measuring the signal intensity.

An array is a solid support with has a hydrophobic surface, allowing the binding of proteins to the surface. Arrays for RPAs and other affinity assays are commercially available and well known to the skilled person in the art. The calibration reagent is immobilized on the array by interaction of the carrier protein with the surface of the array. To avoid unspecific binding to the hydrophobic surface the spotted array preferably is subsequently coated with an unspecific protein, such as e.g. BSA.

The calibration reagent is applied on the array in two or more concentrations. Preferably, the applied concentrations form a dilution series (e.g. a dilution series of 1:2, 1:5, or 1:10). The calibration reagent is preferably applied in at least three different concentrations. More preferably, the calibration reagent is applied in 3 to 20 different concentrations, even more preferred are 5 to 15 different concentrations.

The calibration reagent can be applied at the desired position on the array as a spot. The calibration reagent is typically solved in a buffer. A buffer solution is an aqueous solution consisting of a mixture of a weak acid and its conjugate base or a weak base and its conjugate acid. A suitable buffer is e.g. CSBL spotting buffer (Product number 9020, Zeptosens, Witterswil, Switzerland). In a preferred embodiment said buffer comprises matrix proteins. A preferred matrix protein is BSA, more preferably the matrix protein is acetylated BSA. Typically, the applied calibration reagent solution is allowed to dry before incubating the array with the affinity reagent of interest.

The spots of the calibration reagent on an array are typically arranged in fields. Array fields can form geometrical areas such as e.g. squares, rectangles, circles, and triangles. Examples of an array layout are shown in FIGS. 1B and 12. Spots in two fields can have e.g. different dilution series (different ranges of concentrations). Positive and negative controls are typically arranged in a different field than the calibration reagent.

With the standard curve the concentration of a protein of interest in a sample proceeded in the same way as the calibration reagent can be back calculated.

Therefore, the present invention provides a method for quantifying a protein comprising the epitope of interest in a biological sample comprising

a) immobilizing on an array
i) the above described calibration reagent in two or more concentrations, and
ii) one or more biological samples,

b) incubating said array with a detectable affinity reagent of interest,

c) measuring the signal intensity of the bound affinity reagent for each of the two or more concentrations of the calibration reagent and for each of the one or more biological samples,

d) correlating the signal intensity with the amount of epitope of interest, and

e) quantifying the protein comprising the epitope of interest in the one or more biological samples.

The biological sample is of biological origin and a complex mixture of molecules. A sample can be formed e.g. by lysates of cells, cell extracts, body fluids (e.g. whole blood, serum, plasma, urine, tissue fluid, synovial fluid, tears, urine, saliva, and lymph). The samples may be fractioned or non-fractioned.

The biological sample, like the calibration reagent, is applied at the desired position on the array as a spot. The biological samples may be diluted or undiluted with a buffer. In a preferred embodiment said buffer comprises matrix proteins. A preferred matrix protein is BSA, more preferably the matrix protein is acetylated BSA. Typically, the applied samples are allowed to dry before incubating the array with the affinity reagent of interest.

The spots of the biological samples and the calibration reagent on an array are typically arranged in fields. Array fields can form geometrical areas such as e.g. squares, rectangles, circles, and triangles. Examples of an array layout are shown in FIGS. 1B and 12. Preferably, the spots of the biological samples are arranged in another field than the spots of the calibration reagent.

Preferably, the applied concentrations of the calibration reagent form a dilution series (e.g. a dilution series of 1:2, 1:5, or 1:10). Also preferred is that the calibration reagent is applied in at least three different concentrations. More preferably, the calibration reagent is applied in 3 to 20 different concentrations, even more preferred are 5 to 15 different concentrations. It is well known by the person skilled in the art how to choose the range of concentrations of the calibration reagent near the expected concentration of the peptide of interest in the biological samples and within the working range of the detection method.

The affinity reagent of interest is incubated at least for 30 minutes on the array, preferably, it is incubated on the array for more than 1 hour, more preferably for 1 to 16 hours, most preferably about 12 hours (12 hours±30 minutes). The excess of affinity reagent is removed and preferably the array is washed before measuring the signal intensity.

Furthermore, the present invention provides the use of the above described standard curve for characterizing the affinity reagent by determining the lower limit of detection, the sensitivity and the dynamic range of the affinity reagent of interest.

The term “lower limit of detection (LOD)” refers to minimum amount of the epitope of interest that can be detected with the affinity reagent. The term “dynamic range” of the affinity reagent refers to the measurable range of concentration of the calibration reagent. The dynamic range is typically determined with a standard curve, whereby the dynamic range is the range of calibration reagent concentrations for which there is a linear or substantially linear correlation to the measured signal. The terms “lower limit of detection” and “dynamic range” are well known to the skilled person in the art.

Therefore, the present invention provides a method for determining the lower limit of detection of an affinity reagent of interest comprising

a) immobilizing the above described calibration reagent in two or more concentrations on an array,

b) incubating said array with an detectable affinity reagent of interest,

c) measuring the signal intensity of the bound affinity reagent for each of the two or more concentrations of the calibration reagent,

d) correlating the signal intensity with amount of epitope of interest, and

e) determining the minimum amount of the epitope of interest that can be detected with the affinity reagent.

In a preferred embodiment, the minimum amount of the detectable epitope of interest can be determined with back-calculating the concentrations which correspond to the signal measured at the blank plus three times the standard deviation of the blank. The blank level is the detected signal of a sample which does not comprise the calibration reagent but is otherwise identical with the samples comprising the calibration reagent.

The calibration reagent is applied as described above. Preferably, the applied two or more concentrations form a dilution series (e.g. a dilution series of 1:2, 1:5, or 1:10). Also preferably, the calibration reagent is applied in at least three different concentrations. More preferably, the calibration reagent is applied in 3 to 20 different concentrations, even more preferred are 5 to 15 different concentrations.

The affinity reagent of interest is incubated at least for 30 minutes on the array, preferably, it is incubated on the array for more than 1 hour, more preferably for 1 to 16 hours, most preferably about 12 hours (12 hours±30 minutes). The excess of affinity reagent is removed and preferably the array is washed before measuring the signal intensity.

The present invention provides a method for determining the sensitivity of the affinity reagent of interest comprising

a) immobilizing the above described calibration reagent in two or more concentrations on an array,

b) incubating said array with a detectable affinity reagent of interest, wherein said affinity reagent of interest,

c) measuring the signal intensity of the bound affinity reagent for each of the two or more concentrations of the calibration reagent,

d) correlating the signal intensity with the amount of epitope of interest, and thereby generating a standard curve,

e) determining the linear part of the standard curve and

f) determining the slope of the linear part of the standard curve.

The calibration reagent is applied as described above. Preferably, the applied two or more concentrations form a dilution series (e.g. a dilution series of 1:2, 1:5, or 1:10). Also preferably, the calibration reagent is applied in at least three different concentrations. More preferably, the calibration reagent is applied in 3 to 20 different concentrations, even more preferred are 5 to 15 different concentrations.

The affinity reagent of interest is incubated at least for 30 minutes on the array, preferably, it is incubated on the array for more than 1 hour, more preferably for 1 to 16 hours, most preferably about 12 hours (12 hours±30 minutes). The excess of affinity reagent is removed and preferably the array is washed before measuring the signal intensity.

The present invention provides a method for determining the dynamic range of the affinity reagent of interest comprising

a) immobilizing the above described calibration reagent in two or more concentrations on an array,

b) incubating said array with a detectable affinity reagent of interest, wherein said affinity reagent of interest,

c) measuring the signal intensity of the bound affinity reagent for each of the two or more concentrations of the calibration reagent,

d) correlating the signal intensity with the amount of epitope of interest, and thereby generating a standard curve,

e) determining the linear part of the standard curve and

f) determining the range of concentration of the calibration reagent of interest of the linear part of the standard curve.

The calibration reagent is applied as described above. Preferably, the applied two or more concentrations form a dilution series (e.g. a dilution series of 1:2, 1:5, or 1:10). Also preferably, the calibration reagent is applied in at least three different concentrations. More preferably, the calibration reagent is applied in 3 to 20 different concentrations, even more preferred are 5 to 15 different concentrations.

The affinity reagent of interest is incubated at least for 30 minutes, preferably it is incubated on the array for more than 1 hour, more preferably for 1 to 16 hours, most preferably about 12 hours (12 hours±30 minutes). The excess of affinity reagent is removed and preferably the array is washed before measuring the signal intensity.

In addition, the calibration reagent can be used for determining the specificity of an affinity reagent. The term “specificity” as used herein refers to the selectivity of the affinity reagent for the epitope of interest. An low specific affinity reagent binds also epitopes other than the epitope of interest.

Therefore, the present invention provides a method for determining the specificity of an affinity reagent comprising the following steps:

a) immobilizing on an array
i) the above described calibration reagent comprising the epitope of interest and
ii) at least one sample comprising a control peptide conjugated to protein carrier, wherein the control peptide does not comprise the epitope of interest,

b) incubating the array with a detectable affinity reagent of interest,

c) measuring the signal intensity of the bound affinity reagent on the array, and

d) comparing the signal intensity correlating with the epitope of interest of the calibration reagent with the signal intensity correlating with the control peptide.

Detection of a significant signal means that the antibody of interest has a low specificity as it recognizes also epitopes other than the epitope of interest. The term “significant signal” as used herein is a signal which is significant higher than the background signal, wherein a background signal is the signal detected in the absence of the a sample (e.g. signal detected between the spots). Significant higher means that the difference to the background signal is statistically relevant (p≦0.05, preferably, p≦0.01).

Preferably, the concentration of the control peptide applied per spot on the array is close (+/−5%) to the concentration of the epitope peptide. Spots of the calibration reagent and spots of samples comprising the control peptide having a similar peptide concentration are preferably grouped on the array in fields, whereby the fields can form geometrical areas like for example squares, rectangles, circles, and triangles. The total protein concentration of the samples in two fields can be different (e.g. a high epitope concentration in field 1 and a low epitope concentration in field 2).

The control epitope does not comprise the epitope of interest, but it comprises an epitope which is different from the epitope of interest. This epitope of the control epitope (control epitope) can for example be the modified equivalent of the epitope of interest (e.g. the unphosphorylated equivalent of the epitope of interest). Preferably, more than one sample comprising a control peptide conjugated to protein carrier is applied on the array. The control peptide in these samples can have different concentrations or they can comprise different epitopes. The control epitope in one sample can for example be the un-phosphorylated equivalent of the epitope of interest and in another sample the control epitope has a different amino acid sequence than the epitope of interest.

The affinity reagent of interest is for at least 30 minutes incubated on the array, preferably for at least 1 hour, more preferably for 1 to 16 hours, most preferably about 12 hours (12 hours±30 minutes). The excess of the mixture is removed and preferably the array is washed before measuring the signal intensity.

Alternatively, the detectable affinity reagent of interest is incubated with a free epitope peptide of interest prior to step a) and in step b) the array is incubated with the mixture of the affinity reagent and free peptide.

Therefore, the present invention also provides a method for determining the specificity of an affinity reagent comprising the following steps:

a) incubating a detectable affinity reagent of interest with free epitope peptide of interest,

b) immobilizing on an array
i) the above described calibration reagent comprising the epitope of interest and
ii) a sample comprising a control peptide conjugated to protein carrier, wherein the control peptide does not comprise the epitope of interest,

c) incubating the array with the mixture of the affinity reagent of interest and the free epitope peptide of step a),

d) measuring the signal intensity of the bound affinity reagent on the array and

e) comparing the signal intensity correlating with the epitope of interest of the calibration reagent with the signal intensity correlating with the control peptide.

A “free epitope peptide of interest” is an epitope peptide of interest which is not attached to another molecule. In particular, the free peptide is not attached to a protein carrier.

The concentration of the free peptide is chosen so that the affinity reagent of interest is saturated with the free peptide. This concentration can be determined for example by the following method: a) incubating an detectable affinity reagent of interest with at least to two different concentrations of free epitope peptide, b) immobilizing the above described calibration reagent on at least two arrays, c) incubating the arrays with a mixture of the affinity reagent of interest and the free epitope peptide of step a) wherein each array with a mixture comprising a different concentration of free epitope peptide, d) measuring the signal intensity of the bound affinity reagent on the arrays and determining the concentration of free peptide at which the affinity reagent is saturated with it so that no affinity reagent binds to the array.

The free peptide is preferably incubated with the affinity reagent of interest for at least 30 minutes, preferably for at least 1 hour, more preferably for 1 to 16 hours, most preferably about 12 hours (12 hours±30 minutes).

The mixture of affinity reagent of interest and free peptide is for at least 30 minutes, preferably incubated on the array for at least 1 hour, more preferably for 1 to 16 hours, most preferably about 12 hours (12 hours±30 minutes). The excess of the mixture is removed and preferably the array is washed before measuring the signal intensity.

Having now generally described this invention, the same will become better understood by reference to the specific examples, which are included herein for purpose of illustration only and are not intended to be limiting unless otherwise specified, in connection with the following figures.

### FIGURES

FIG. 1A shows the structure of a tagged calibration reagent. (A) Peptide comprising the epitope of interest, (B) Hydrophilic linker, (C) Carrier protein, (D) Label for concentration determination of the calibration reagent (e.g. Dabsyl). The peptide is covalently bound to the linker and the linker is covalently bound to the protein carrier.

FIG. 1B shows a schematic array layout. The array is divided into 12 Array fields (number 1-12 in squares) and a Control field (1-16). Each array field comprises the 12 sample positions (3 rows×4 positions, each in duplicate spots=>24 spots) of a complete standard dilution series (position 1-12). Arrows indicate the direction of decreasing concentration. Two adjacent array fields are arranged in mirror position, to avoid that spots of high standard concentration meet spots of low standard concentration. The Control field was used to co-array lysate controls (16 sample positions in duplicate spots).

FIGS. 2A and 2B shows the peptide sequences of human Erk1 (FIG. 2A) and human Erk2 (FIG. 2B) proteins. Selected peptide sequence around the phosphorylation sites in the center of the two proteins is underlined (identical for both proteins). Selected peptide sequence (peptide comprising the epitope) for total Erk1 (BioSource antibody) is marked with framed. Different amino acids in the corresponding sequence of Erk2 protein are indicated by arrows. FIG. 2C shows the preferred positions of phosphorylated amino acids (A . . . (p)) in a peptide comprising the epitope of interest. FIG. 2D shows a schematic representation of a standard curve whereby the dynamic range (dr) is indicated. C=concentration of the calibration reagent, S=signal intensity.

FIG. 3 shows assay image sections of arrays containing printed standard curves of peptide-BSA reagents of different peptide-protein conjugate ratios and probed with antibodies against the epitope. FIG. 3A: Histone H3-BSA, ratio: 0.7× (I), 2.7× (II), 13.4 (III), Antibody: 1:5000 Abcam ab1791. FIG. 3B: pRb-BSA, ratio: 0.25× (I), 1× (II), 10× (III), Antibody: 1:500 CST no 9308. FIG. 3C: pErk1/2-BSA, ratio: 0.7× (I), 2.7× (II), 13.4× (III), Antibody: 1:500 CST no 9101. Array layout (AL): The standard curves were printed as 12 serial dilution curves (2-fold dilutions), each dilution as duplicate spots. Start concentrations of the different peptide-BSA reagents were adjusted to a uniform epitope concentration of 50 nM (spot 1).

FIG. 4 shows a quantitative assay signals analyzed from standard curves of printed HistoneH3-BSA 2.7× reagent (Histone H3 assay, 1:5000) (FIG. 4A-C) and printed pErk-BSA 2.7× reagent (pErk1/2 assay, 1:500) (FIG. 4D-F). Solid circles represent the measured standard curve signals as mean of duplicate spots, the solid line represents the fit curve of the 1-site binding model; squares indicate the signals of corresponding co-arrayed lysate controls, total protein concentration of the lysates: 0.25 mg/ml (neg=negative and pos=positive treated control, number indicates the identity of the lysate (see table 1)). FIG. 4A and 4D: lin-log curves; FIG. 4C and FIG. 4F: log-log curves. FIG. 4B and FIG. 4E show assay images with specific epitope binding responses. The standard curves were printed as 12 serial dilution curves (2-fold dilutions), each dilution as duplicate spots. Start concentrations of the different peptide-BSA reagents were adjusted to a uniform epitope concentration of 50 nM (spot 1). Both cases show a dynamic range of about 4 orders of magnitude in concentration and signal range within one image.

FIG. 5A shows an effect of increasing additions of matrix protein (acBSA) to print solutions of standard curve reagents, shown for the case of pErk1/2 assay (1:500). (Left side a,b,c) printed pErk-BSA 2.7× reagent; (right side d,e,f,) printed recombinant Erk1 protein (Invitrogen). (Top a,d) Dilution series printed in pure spotting buffer, (mid b,d) in spotting buffer plus 50 μg/ml acBSA, and (bottom) in spotting buffer plus 100 μg/ml acBSA. acBSA=acetylated BSA. The addition of acBSA led to more homogenous spot morphology.

FIG. 5B shows the effect that the type of matrix protein (acBSA vs BSA) has which is added to print solutions, shown for the case of Histone H3 assay. (Left side a,b) Dilution series of Histone-BSA 2.7× reagent; (right side) dilution series of recombinant Histone H3 protein (Roche). No major signal difference was detected, but acBSA addition led to more homogeneous spot morphology.

FIG. 6 shows array signal images of Histone H3 assay (1:10′000 dilution of ab179lantibody) in absence (normal assay: A)) and presence (competition assay: B to D) of increasing concentrations (B: 100 nM, C: 1000 nM; D: 10′000nM) of corresponding free epitope peptide in antibody solution. Specific antibody signals of standards/lysate controls in the normal assay were ˜completely/completely suppressed by the competition reaction at the highest concentration of free peptide (10000 nM). Exposure time: 0.5 s and Display Range (DR) of images 0 . . . 10000.

FIG. 7 shows standard curves for Histone H3 assay (1:10000 Abcam ab1791) from 12 point dilutions curves of Histone H3 peptide standard Histone H3-BSA 2.7× (solid circles) and Histone H3 recombinant protein from Upstate with most prominent signals (solid triangles). Signals of control lysates (250 μg/ml) were added to the peptide standard curves for comparison (solid squares); concentrations were back-calculated from signals. Graphs show mean signals of printed standard dilutions (solid data points) and the fit curve of a one site binding model (solid line, Hill fit). Data points correspond to mean signals of duplicate spots, error bars indicate their standard deviations. LOD values were back-calculated concentrations from the fit curve at mean blank signal level plus 3-fold standard deviation (see dotted lines in the right graphs: - - - - for Histone H3 peptide standard, [symbol] for Histone H3 recombinant protein from Upstate). FIG. 7A: Assay 1, Lin-Log plot, LOD (Histone H3 protein)=0.133 nM, LOD (Histone H3-BSA 2.7×)=0.104 nM. FIG. 7B: Assay 1, Log-Log plot. FIG. 7C: Assay 2, Lin-Log plot, LOD (Histone H3 protein)=0.178 nM, LOD (Histone H3-BSA 2.7×)=0.141 nM. FIG. 7D: Assay 2, Log-Log plot. (correlation coefficients of r2 >0.99).

FIG. 8 shows standard curves for pRb assay (1:250 CST #9308) from 12 point dilutions curves of pRb peptide standard (solid circles) and pRb recombinant protein from Active Motif (solid triangles). Signals of control lysates (400 μg/ml) were added to the peptide standard curves for comparison (solid squares, pos=positive, neg=negative, number indicates identity of lysate, see table 1); concentrations were back-calculated from signals. Graphs show mean signals of printed standard dilution (solid data points) and the fit curve of a one site binding model (solid line, Hill fit). Data points correspond to mean signals of duplicate spots; error bars indicate their standard deviations. LOD values were back-calculated concentrations from the fit curve at mean blank signal level plus 3-fold standard deviation (see dotted lines in the right graphs: - - - - for Rb peptide standard, [symbol] for Rb recombinant protein from Active Motif). FIG. 8A: Lin-Log plot of Assay 1, LOD (pRb protein)=0.117 nM, LOD (pRb-BSA 1×)=0.024 nM. FIG. 8B: Assay 1, Log-Log plot. FIG. 8C: Assay 2, Lin-Log plot, LOD (pRb protein)=0.077 nM, LOD (pRb-BSA 1×)=0.026 nM. FIG. 8D: Assay 2, Log-Log plot. Correlation coefficients of r²>0.99

FIG. 9 shows standard curves for pErk1/2 assay (1:500 CST #9101) from 12 point dilutions curves of pErk1/2 peptide standard pErk1-BSA 2.7× (solid circles) and pErk1 recombinant protein from Invitrogen with most prominent signals (solid triangles). Signals of control lysates (400 μg/ml) were added to the peptide standard curves for comparison (solid squares, pos=positive, neg=negative, number indicates identity of lysate, see table 1); concentrations were back-calculated from signals. Graphs show mean signals of printed standard dilution (solid data points) and the fit curve of a one site binding model (solid line, Hill fit). Data points correspond to mean signals of duplicate spots; error bars indicate their standard deviations. LOD values were back-calculated concentrations from the fit curve at mean blank signal level plus 3-fold standard deviation (see dotted lines in the right graphs: - - - - for Erk1/2 peptide standard, [symbol] for Erk1/2 recombinant protein from Invitrogen). FIG. 9A: Assay 1 (Lin-Log plot), LOD (pErk1 protein)=0.058 nM, LOD (pErk1-BSA2.7×)=0.028 nM. FIG. 9B: Assay 1 (Log-Log-plot). FIG. 9C: Assay 2 (Lin-Log plot), LOD (pErk1 protein)=0.052 nM, LOD (pErk1-BSA2.7×)=0.032 nM. FIG. 9D: Assay 2 (Log-Log-plot). Correlation coefficients of r²>0.99

FIG. 10 shows Standard curves for Erk1/2 assay (1:1000 Biosource 44-654G) from 12 point dilutions curves of Erk1 peptide standard Erk1-BSA 2.7× (solid circles) and Erk1 recombinant protein from Invitrogen with most prominent signals (solid triangles). Total Erk signals of pErk control lysates (400 μg/ml) were added to the peptide standard curves for comparison (solid squares, pos=positive, neg=negative, number indicates identity of lysate, see table 1); concentrations were back-calculated from the signals. Graphs show mean signals of printed standard dilution (solid data points) and the fit curve of a one site binding model (solid line, Hill fit). Data points correspond to mean signals of duplicate spots; error bars indicate their standard deviations. LOD values were back-calculated concentrations from the fit curve at mean blank signal level plus 3-fold standard deviation (see dotted lines in the right graphs). FIG. 10A: Lin-Log plots of Assay 1, LOD (Erk1 protein) =0.059 nM, LOD (Erk1-BSA2.7×)=0.045 nM. FIG. 10B: Log-Log-plots of Assay 1. FIG. 10C: Lin-Log plot of Assay 2 (bottom): LOD (Erk1 protein)=0.084 nM, LOD (Erk1-BSA2.7×)=0.046 nM. FIG. 10D: Assay 2,Log-Log-plot. Correlation coefficients of r²>0.99

FIG. 11 shows Standard curves for Erk1/2 assay (1:1000 Biosource 44-654G) from 12 point dilutions curves of Erk1 peptide standard Erk-BSA 2.7× (solid circles) and pErk1 recombinant protein from Invitrogen with prominent signals comparable to total Erk1 protein from Invitrogen (solid diamonds). Total Erk signals of pErk control lysates were added to the peptide standard curves for comparison (solid squares); concentrations were back-calculated from the signals. Graphs show mean signals of printed standard dilution (solid data points) and the fit curve of a one site binding model (solid line, Hill fit). Data points correspond to mean signals of duplicate spots; error bars indicate their standard deviations. LOD values were back-calculated concentrations from the fit curve at mean blank signal level plus 3-fold standard deviation (see dotted lines in the right graphs). FIG. 11A: Assay 1 (Lin-Log plot), LOD (pErk1 protein)=0.040 nM, LOD (Erk1-BSA2.7×)=0.045 nM. FIG. 11B: Assay 1 (Log-Log-plot). FIG. 11C: Assay 2 (Lin-Log plot), LOD (pErk1 protein)=0.047 nM, LOD (Erk1-BSA2.7×)=0.046 nM. FIG. 11D: Assay 2 (Log-Log-plot). Correlation coefficients of r²>0.99.

FIG. 12 shows the Array layout for spiking experiments of Example 5. Conditions of printed standard dilution series, applied reagents and spiked lysates are given in Table 7.

FIG. 13 shows Array signal images of Histone H3 assay (1:10′000 dilution of ab1791antibody). Duplicate assay 1 (A1) and assay 2 (A2) as well as blank assay (B) are shown. Exposure time: 1 s and Display Range (DR) of images: 300 . . . 15000. Assays were performed with Histone H3 antibody (Abcam, ab1791) at 1:10′000 dilution.

FIG. 14 shows Standard curves for Histone H3 assay (1:10000 Abcam ab1791): 8 point dilutions curves of Histone H3 peptide standard (Histone H3-BSA 2.7×) in buffer (solid circles) and 7 point dilution series of Histone H3 peptide standard spiked into control HistoneH3(-) lysate 6 (solid triangles). Shown signals of the spike-in curves were corrected for the signal contribution of the endogenous concentration of Histone H3 of the pure lysate (values of offset (blank) signals and of back-calculated endogenous protein concentrations are indicated in the graphs). Graphs show the measured data points (solid data points) and the fit curves of a one site binding model (solid line, Hill fit). Data points correspond to the mean signals of N=5 replicate spots per concentration, error bars indicate their standard deviations. Assay 1 (top) and Assay 2 (bottom). Lin-Log plots (left side) and Log-Log plots (right side). Correlation coefficients is r²>0.99.

FIG. 15 shows Array signal images of pRb assay (1:500 dilution of CST #9308 antibody). Duplicate assay 1 (A1) and assay 2 (A2) as well as blank assay (B) are shown. Exposure time: 16 s and Display Range (DR) of images: 1500 . . . 30000. Assays were performed with pRb antibody (CST #9308) at 1:250 dilution.

FIG. 16 shows Standard curves for pRb assay (1:250 CST #9308): 8 point dilutions curves of pRb peptide standard (pRb-BSA 1×) in buffer (solid circles) and 7 point dilution series of pRb peptide standard spiked into pRB(-) lysate 12 (solid triangles). Shown signals of the spiked-in curves were corrected for the signal contribution of the endogenous pRb concentration of the pure lysate (values of offset (blank) signals and of back-calculated endogenous pRb concentrations are indicated in the graphs). Graphs show the measured data points (solid data points) and the fit curves of a one site binding model (solid line, Hill fit). Data points correspond to the mean signals of N=5 replicate spots per concentration, error bars indicate their standard deviations. Assay 1 (top) and Assay 2 (bottom). Lin-Log plots (left side) and Log-Log plots (right side). Correlation coefficients is r²>0.99.

FIG. 17 shows array signal images of pErk1/2 assay (1:500 dilution of CST #9101 antibody). Duplicate assay 1 (A1) and assay 2 (A2) as well as blank assay (B) are shown. Exposure time: 2 s and Display Range (DR) of images: 500 . . . 30000. Assays were performed with pErk1/2 antibody (CST #9101) at 1:500 dilution.

FIG. 18 shows Standard curves for pErk1/2 assay (1:500 CST #9101): 8 point dilutions curves of pErk1/2 peptide standard (pErk1-BSA 2.7×) in buffer (solid circles) and 7 point dilution series of pErk1/2 peptide standard spiked into pErk(-) lysate 13 (solid triangles). Shown signals of the spiked-in curves were corrected for the signal contribution of the endogenous pErk1/2 concentration of the pure lysate (values of offset (blank) signals and back-calculated endogenous protein concentrations are indicated in the graphs). Graphs show the measured data points (solid data points) and the fit curves of a one site binding model (solid line, Hill fit). Data points correspond to the mean signals of N=10 replicate spots per concentration, error bars indicate their standard deviations. Assay 1 (top) and Assay 2 (bottom). Lin-Log plots (left side) and Log-Log plots (right side).

FIG. 19 shows Array images of Erk1/2 assay (1:1000 dilution of BioSource 44-654G antibody). Duplicate assay 1 (A1) and assay 2 (A2) as well as blank assay (B) are shown. Exposure time: 2 s and Display Range (DR) of images: 500 . . . 30000.

FIG. 20 shows Standard curves for Erk1/2 assay (1:1000 Biosource 44-654G): 8 point dilutions curves of pErk1/2 peptide standard pErk1-BSA 2.7× in buffer (solid circles) and 7 point dilution series of pErk1/2 peptide standard spiked into pErk(-) lysate 13 (solid triangles). Graphs show the measured data points (solid points) and the mean signals of all data points (solid lines). The Erk1/2 assay generated, as expected, almost zero signals for the pErk1/2 peptide standard dilutions in buffer, and uniform prominent signals for all lysate spots spiked with different concentrations of pErk1/2. The mean signals represent the endogenous Erk1/2 levels of lysate 13 independent on spike-in concentration. Signal axes were scaled as for the pErk1/2 assay (see FIG. 19). Data points correspond to the mean signals of N=10 replicate spots for each spike-in concentration, error bars indicate their standard deviations. Assay 1 (left): mean signal (standard curve)=0.010±0.002 RFI, mean signal (spike curve)=1.097±0.057 RFI and Assay 2 (right): mean signal (standard curve)=0.008±0.002 RFI, mean signal (spike curve)=0.969±0.016 RFI.

### EXAMPLES

Commercially available reagents referred to in the examples were used according to manufacturer's instructions unless otherwise indicated.

Example 1

### Materials and Methods

Lysate Samples

Protein concentrations were determined in a modified Bradford test (Coomassie Plus Protein Assay Reagent, no. 23238, Pierce). The lysate samples were stored in the freezer at −70° C. until use.

TABLE 1
Control lysate samples.

| | | Protein | | |
|---|---|---|---|---|
| | | detected: | | Protein conc. |
| Lysate# | Protein ( ) | yes/no | Cell line | [mg/ml] |
| 5 | Histone H3 | yes | HCT116 | 2.5 |
| 6 | Histone H3 | no | HCT116 | 1.5 |
| 7new | Erk 1/2p(Thr202/Tyr204) | yes | Hela | 9.6 |
| 8new | Erk 1/2p(Thr202/Tyr204) | no | Hela | 11.6 |
| 9new | Erk 1/2p(Thr202/Tyr204) | no | HCT116 | 3.7 |
| 10new | Rb p(Ser807/Ser811) | yes | Hela | 11.5 |
| 12 | Rb p(Ser807/Ser811) | no | Hela | 10.2 |
| 13 | Rb p(Ser807/Ser811) | no | HCT116 | 3.0 |

Rb = Retinoblastoma tumor suppressor protein,
Erk = Extracellular signal-regulated kinase,
p = phosphorylated amino acid residue

For array printing in the different working packages, the lysate samples were adjusted to a given protein concentration in CLB1 (lysis buffer) (Zeptosens) and finally diluted 1:10 in CSBL spotting buffer (Zeptosens)). The final printed protein concentrations are always indicated in the respective sections.

Reverse Phase Protein Microarrays Array Printing

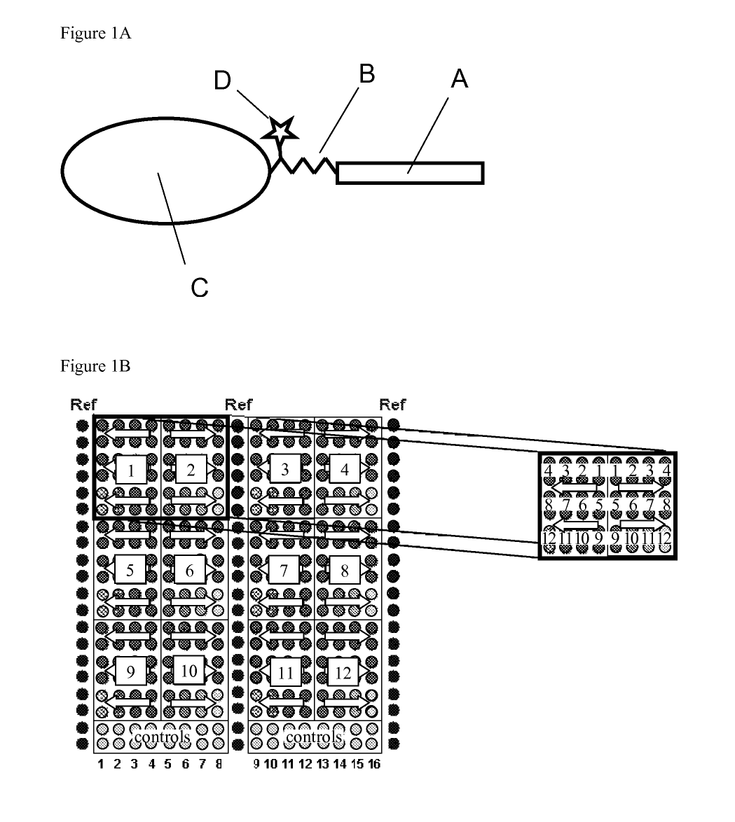

The typical array layout is depicted in FIG. 1B. Each array contained 19×20 (380) spots. 320 spots were used for 160 sample positions, each to be printed in duplicate spots. Spot diameters were about 150 μm. The spot-to-spot distance was 280 μm in horizontal axis and 300 μm in vertical axis.

The array was divided into 12 array fields. Each field comprised 12 sample positions, each position printed in duplicate spots. The 12 sample positions were arranged as 3 rows of 4 positions each. 12-point dilutions series (2-fold dilutions) were printed in the order of position 1 (highest concentration=start concentration) to position 12 (lowest concentration), see FIG. 1B. The arrows indicate the direction of decreasing concentrations. Adjacent array fields were arranged in mirror position. This was done to avoid that spots of highest standard concentrations met spots of lowest standard concentrations. Lysates were co-arrayed as controls in the Control field (16 positions in duplicate spots).

Arrays for the different work packages were printed in series of replicates (6 arrays per chip) in a number sufficient to perform all experiments. Print solutions for each series were prepared freshly in 384 well plates by means of a liquid handling robot (Tecan Genesis RSP100). For each standard curve, a stock solution of standard reagent at the start concentration (e.g. 50 nM) was prepared. The different samples (12×2-fold dilutions) were prepared as serial dilutions in the plate wells. The volume per well was 25 μl. For printing of control lysates, samples were adjusted to uniform starting concentration (e.g. 1.5 mg/ml) and diluted 1:10 in spotting buffer CSBL (e.g. final concentration=150 μg/ml).

Each spot was arrayed as a single droplet of about 400 picoliter volume, using a commercial piezo-electric arrayer (NanoPlotter NP2, GeSim GmbH, D-Groβerkmannsdorf). Together with the dilution series and lysate samples, a reference material consisting of fluorescence-labeled protein was co-arrayed into three separate rows of landing marks (see FIG. 1B). These reference spots (Ref) were used to compensate for eventual local inhomogeneities of array illumination, array-to-array and chip-to-chip variations. Arrays were produced under clean-room conditions.

Samples for dilution series and lysate controls were always prepared freshly from frozen stocks.

After spotting, the microarrays were blocked with BSA, thoroughly washed with ddH₂O, dried under a nitrogen stream and stored in the dark at +4° C. until use. For the measurements, a fluidic structure is attached to the chip to address each of the 6 identical arrays of a chip individually with analyte-specific antibody solution at the respective assay condition (the chamber volume per array was about 15 μL).

Antibodies and Assay Reagents

Table 2 lists the proteins and corresponding antibodies used in this study.

TABLE 2
List of protein analytes and antibodies.

| | | | antibody | order | | | conc. |
|---|---|---|---|---|---|---|---|
| # | antigen | activation site | supplier | no. | species | lot | [mg/ml] |
| 1 | Histone H3 | | Abcam | ab1791 | rb | n.a. | n.a. |
| 2 | Rb phospho (pRb) | Ser807/811 | CST | 9308 | rb | 8 | 0.050 |
| 3 | p44/42 MAPK | Thr202/Tyr204 | CST | 9101 | rb | 21 | 0.131 |
| | phospho (pERK1/2) | | | | | | |
| 4 | p44/42 MAPK (Erk1/2) | | Biosource | 44-654G | rb | 136 8666A | 1.000 |
| 5 | p44/42 MAPK (Erk1/2) | | CST | 4695 | rb monocl. | 4 | 0.110 |
| 6 | p44/42 MAPK (Erk1/2) | | CST | 9102 | rb | 18 | 0.111 |

NMI-TT provided all other reagents, e.g. labeled detection reagents, buffers, needed to perform the assays on Reverse Phase Protein Arrays (RPA).

Anti-species Fab fragments were used as detection reagents for assay signal generation on the micro arrays.
Alexa Fluor 647 labeled anti-rabbit IgG Fab molecules (Z-25308, Molecular Probes), to detect rabbit polyclonal antibodies bound to the respective analyte

Assay Buffer (Antibody)

The assay buffer for RPA measurements (assay buffer) was 50 mM imidazole/HCl, 150 mM NaCl, 0.1% Tween20, 0.005% sodium azide, pH7.4 with addition of 5% (w/v) BSA

Print Buffers (Calibration Reagent)

The print buffer was CSBL (Zeptosens—a Division of Bayer Schweiz AG).

The following reagents were used as additions during the study: BSA (#T844.2, Roth)
BSA acetylated (#05491, Fluka)

Reverse Phase Protein Arrays—Assay Procedure and Data Analysis

The detection of the protein analyte on the array was performed in a direct two-step sequential immunoassay. The first step comprised the addition of analyte-specific antibody in assay buffer onto the microarray and incubation for over night at 25° C. After removal of excess antibody by washing with assay buffer, the microarrays were incubated with fluorescence-labeled anti-species Fab fragment for 1 hour at 25° C. in the dark. For the detection of the rabbit antibodies applied in this study, Fab fragments at a 500-fold dilution in assay buffer were used. Finally, the arrays were washed and imaged in solution (assay buffer) with the ZeptoREADER® imager instrument (Zeptosens).

Additional competition experiments were performed to test the specificity of antibody-antigen binding in solution and on the array spot. For this, free synthesized peptide product (specific binding epitope sequences for the respectively applied antibodies) was mixed together with primary antibody in assay buffer solution and incubated for 30 min at room temperature, before the reaction mixture was incubated on the array. All other assay steps were performed at conditions comparable to the normal assay described above. The concentrations of the free peptide were chosen in molar excess of the applied antibody concentration (see also Table 2). The peptide concentrations for competition were typically chosen at 1000 nM, 100 nM and 10 nM, if not stated otherwise.

The ZeptoREADER® is a bench top solution for automatic high throughput readout of microarrays. Shortly, up to 36 microarrays (6 chips) can be mounted into one carrier (MTP footprint format). An integrated stacker allows the unattended readout up to 360 microarrays (10 fully loaded carriers) in a single run. Microarrays can be excited at 532 nm (green) and 635 nm (red); fluorescence emission is detected with emission filters passing between 547-597 nm (green) and 650-700 nm (red). For this study, a series of typically 9 fluorescence images for each array was taken in the red detection channel at exposure times in the range of 0.5-16 seconds and stored in a 16 bit tif format for further analysis with ZeptoVIEW™ PRO software (Zeptosens).

Microarray Analysis

Microarray images were analyzed using the software ZeptoVIEW™ Pro 2.0 (Zeptosens). The spot diameter of the array analysis grid, which was aligned to the microarrays, was set constant at 160 μm.

The data analysis for each measurement was performed as follows:
Selection of one analyte image of adequate exposure time (all spot signals below saturation of the image).
Calculation of background-corrected, referenced mean signal intensities for each individual spot (in RFI=Referenced Fluorescence Intensity units). The referenced signal is calculated as the ratio of local sample and reference spot signal.
For all replicates spots of one print condition (in most cases duplicate spots), the background-corrected, referenced mean intensities (RFIs) of each duplicate spot pair were averaged. Signals in the diagrams and tables represent the respective mean signals, error bars correspond to the standard deviations. Numbers of applied replicate spots (N) for averaging are indicated.

In addition, Blank assay experiments in the absence of analyte-specific primary antibody were performed to control for possible non-specific binding contributions of the secondary detection reagents. The RFI signals of all blank images were negligible low (for standards as well as lysate samples) and therefore were not considered in the data analysis process.

Data points of the dilution curves (mean signals of duplicate spots) were fitted using the Excel Add-in software package XLfit v4.3.0 (IDBS, Guildford UK). A one-site binding site model was chosen for the fitting (fit function #251: =D+((Vmax*(x̂n))/((x̂n)+(Km̂n))) with D=signal offset, Vmax=saturation signal, Km=affinity constant, n=1 binding site).

Limits-of-Detection (LODs) were as standard concentrations back-calculated from the fit at mean blank signal (4 lowest data points) plus 3-fold standard deviation.

### Example 2

### Production of Calibration Reagents (Peptide-Protein Conjugates)

Peptide Sequences

Four antigens were selected to be investigated. These antigens were Histone H3, Rb phosphorylated, Erk1/2 phosphorylated and Erk1. The 4 peptide sequences represent the linear binding epitopes of the 4 selected antigens to respectively chosen antibodies. Epitope sequence information was obtained from the antibody vendors. Antigens, epitope amino acid sequences, lengths, epitope position of the antigens and respective antibody information are summarized in Table 3.

TABLE 3
List of antigens, epitope peptide sequences and corresponding
antibody information.

| | | | | Antigen | Vendor of |
|---|---|---|---|---|---|
| # | Antigen | Peptide Sequence | Length | position | Antibody/no |
| 1 | Histone H3 | IQLARRIRGERA | l2mer | 124-135 | Abcam/ |
| | | (SEQ. ID NO: 1) | | | ab1791 |
| 2 | Rb phospho | GNIYIS(p)PLKS(p)PYKIS | l5mer | 802-816 | Cell Signaling |
| | (Ser807/811) | (SEQ. ID NO: 2) | | | Tech./9308 |
| 3 | Erk1/2 | DHTGFLT(p)EY(p)VATRWY | l5mer | 196-210 | Cell Signaling |
| | p44/p42 MAPK | (SEQ. ID NO: 3) | | | Tech./9101 |
| | phospho | | | | |
| | (Thr202/Tyr204) | | | | |
| 4 | Erk1 p42 MAPK | KRITVEEALAHPYLEQYYDPT | 23mer | 317-339 | BioSource/ |
| | | DE | | | 44-654G |
| | | (SEQ. ID NO: 4) | | | |

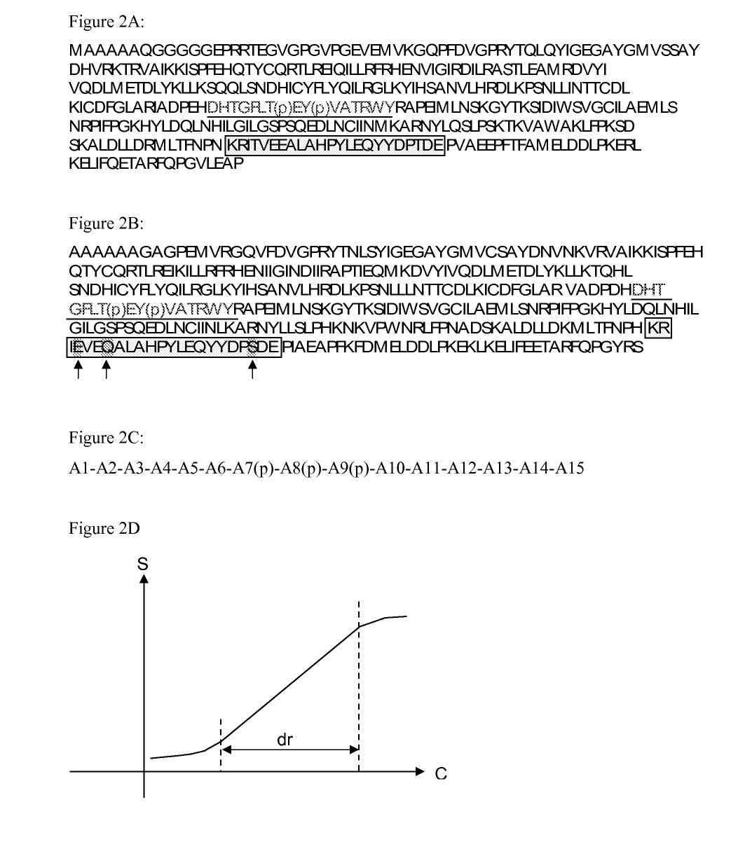

Antibodies against the phosphorylation sites of human Erk1 (p44 MAPK) and Erk2 (p42 MAPK), positioned in the center of the protein, share the same epitope amino acid sequence around amino acid position Thr202 and Tyr204. The antibody against the total form of Erk1 MAPK, here selected from BioSource, was raised against a different linear sequence region positioned at the C-terminal end of the protein. This sequence region is often used also for antibodies of other vendors. The complete peptide sequences of the two proteins Erk1 (SEQ. ID NO: 5) and Erk2 (SEQ. ID NO: 6) are shown in FIG. 2A and 2B. The selected epitope sequence used in this study for Erk1 protein (position 317-339) is homologous to the corresponding C-terminal Erk2 sequence, except for the differences of amino acids in three positions.

Peptide Synthesis

For each of the four selected antigens, two peptides have been synthesized at and by NMI-TT. The two peptides comprised (i) a free peptide form to be used as competition reagents in the immunoassays and (ii) a functionalized form to be used for conjugation to BSA proteins as standard reagent molecules on RPA chips. The functionalized peptides were synthesized with a N-terminal Cys-spacer function for covalent coupling to serum albumin protein using capping cycles. Doa-Doa (Doa=8-Amino-3,6-Dioxaoctanoic acid) was chosen as a C18 length equivalent (PEG-like) hydrophilic spacer. Capping cycles were used in the synthesis to achieve a good specificity and final enrichment of the right target sequences for the protein conjugation. After synthesis, peptides were quality controlled by HPLC for good purity, and mass spectrometry (MS) for the correct molecular mass. Sequence information of the synthesized peptide products with corresponding mass information and achieved purity are summarized in Table 4.

TABLE 4
List of 8 peptide products synthesized at and by NMI-Technologietransfer GmbH
(Reutlingen, Germany). For each antigen, a free form of the peptide was
synthesized as competition reagent, and a functionalized form for covalent
conjugation to BSA protein (standard reagent). The functionalized form of the
peptides were synthesized with an appended N-terminal spacer Dabs-Cys(C)-Doa-
Doa. Dabs = Dabsyl (absorbance label). Doa = 8-Amino-3,6-Dioxaoctanoic acid
(hydrophilic spacer). Cys (C) was used as functional group for covalent
coupling the peptide to BSA via thiol chemistry. Peptides were synthesized
as N-terminal free (H2N) or acetylated forms (Ac), and C-terminal free (COOH)
or amidated forms (CONH2). Phosphorylated amino acids are marked by bold
letters. (funct. = functionalized, phospho, p = phosphorylated)

| | | | | Found | HPLC |
|---|---|---|---|---|---|
| | | | Mass | mass | purity |
| Antigen | Peptide sequence | Form | (Da) | (Da) | (%) |
| Histone | Ac-IQLARRIRGERA-COOH | Free | 1479.87 | 1480.10 | >99 |
| H3 | Dabs-C-Doa-Doa-IQLARRIRGERA-COOH | funct. | 2118.09 | 2118.49 | 99.7 |
| Rb | H2N-GNIYIS(p)PLKS(p)PYKIS-CONH2 | Free | 1837.88 | 1837.90 | 95.4 |
| phospho | Dabs-C-Doa-Doa-GNIYIS(p)PLKS(p)PYKIS-CONH2 | funct. | 2518.11 | 2518.56 | 96.9 |
| Erk1/2 | Ac-DHTGFLT(p)EY(p)VATRWY-CONH2 | Free | 2058.83 | 2058.68 | 95.0 |
| phospho | Dabs-C-Doa-Doa-DHTGFLT(p)EY(p)VATRWY-CONH2 | funct. | 2697.05 | 2696.88 | >99 |
| Erk1 | Ac-KRITVEEALAHPYLEQYYDPTDE-CONH2 | Free | 2820.36 | 2820.15 | >99 |
| | Dabs-C-Doa-Doa-KRITVEEALAHPYLEQYYDPTDE- | funct. | 3460.85 | 3460.35 | 99.0 |
| | CONH2 | | | | |

All peptides reached a high purity of >95% as specified (most of them >99%) and correct molecular mass. No major difficulties were encountered during the synthesis of these peptides.

Peptide-Protein Conjugation

Final standard reagents were produced as respective peptide-protein conjugates. For this, each functionalized form of the peptide was conjugated in molar excess at 3 different ratios (rations are indicated in Table 5) to pre-activated (maleimide-activated) bovine serum albumin (BSA) via covalent coupling to its free N-terminal Cys group, 2 mg of protein were used for each coupling reaction. The peptide coupling was described earlier in Poetz el al., Proteomics 5, 2402-2411 (2005). Shortly, the solid peptides were dissolved as concentrated stocks in 100% DMSO and were subsequently diluted to working concentrations in PBS pH 7.4 buffer containing DMSO at a maximum of 20%. Peptide and activated BSA solutions were mixed and incubated in the dark for 2 h at room temperature. Unconjugated peptide was removed by means of a spin column (size exclusion) and fractions of the conjugate proteins were collected in PBS pH7.4. Subsequently, the (coupled) peptide concentrations of the peptide-protein fractions were determined by spectrophotometric absorbance measurement at 466 nm (maximum of spectral Dabsyl absorbance, extinction coefficient 33′000 M⁻¹ cm⁻¹, one label per peptide). The absorbance color of the peptide-protein fractions was clearly visible by eye (see FIG. 2).

The protein concentrations of the conjugate fractions were determined according to Bradford. Total protein concentrations were around 1.5 mg/ml. Measured peptide concentrations, total protein concentrations and final calculated peptide:protein (dye:protein) ratios of formed products are summarized in Table 5.

TABLE 5
12 standard reagents produced as peptide-protein conjugates.

| | | | | | | | | Ratio |
|---|---|---|---|---|---|---|---|---|
| Preparation | | | Peptide concentration | | | Protein concentration | | Peptide:Protein |
| | molar excess | | absorbance | net | Dabs (D) | Bradford | Prot. Conc | D:P |
| Conjugate | peptide:protein | buffer | 466 nm | absorbance | conc (μM) | mg/ml | (P) μM | ratio |
| BSA control | | PBS | −0.003 | 0 | 0 | | (pure BSA) | |
| BSA-HistoneH3 | 13.4 x | PBS | 0.264 | 0.267 | 81 | 1.92 | 29 | 3.10 |
| | 2.7.x | PBS | 0.040 | 0.043 | 13 | 2.16 | 32 | 0.41 |
| | 0.7 x | PBS | 0.004 | 0.006 | 2 | 1.67 | 25 | 0.08 |
| BSA-pRb | 10 x | PBS | 0.061 | 0.063 | 19 | 1.12 | 17 | 1.19 |
| | 1 x | PBS | 0.033 | 0.035 | 11 | 1.74 | 26 | 0.41 |
| | 0.25 x | PBS | 0.001 | 0.004 | 1 | 1.80 | 27 | 0.05 |
| BSA-pERK1/2 | 13.4 x | PBS | 0.363 | 0.366 | 111 | 1.48 | 22 | 6.30 |
| | 2.7x | PBS | 0.071 | 0.074 | 22 | 1.71 | 26 | 0.89 |
| | 0.7x | PBS | 0.018 | 0.021 | 6 | 1.49 | 22 | 0.27 |
| Add-On preparation | | | | | | | | |
| BSA-Erk1 | 13.4 x | PBS | 0.463 | 0.444 | 134 | 2.02 | 33 | 5.17 |
| | 2.7.x | PBS | 0.113 | 0.094 | 28 | 1.95 | 29 | 1.01 |
| | 0.7 x | PBS | 0.032 | 0.013 | 4 | 1.30 | 19 | 0.21 |

For each antigen, 3 variants of peptide-proteins conjugates were prepared comprising different peptide:protein molar ratios. Conjugated peptide concentration was determined via photometric absorbance measurement of the peptide-integrated Dabsyl label using activated unlabeled protein (BSA) as a control. Total protein concentration of conjugated product was determined via a Bradford test. Peptide:Protein ratio was calculated as molar dye:protein ratio, corrected for mass addition of the conjugated peptides.

Quality control of the peptide conjugates was performed via SDS-PAGE (4-12% gel) using the pure pre-activated BSA as a reference. Gels were Coomassie stained for 60 min. The gel images showed pure product bands as expected, with mass shifts corresponding to the different calculated peptide:protein coupling ratios. Typically, finally determined coupling ratios reached 17-40% of the initially prepared molar excess ratios of peptide:protein, which was according to our previous experience with other peptides. The variations may be due to different solubilities at the applied high starting concentrations and/or e.g. different peptide conformational structures of the peptides in the aqueous coupling buffer.

All four peptide-protein conjugate standard reagents were pure according to PAGE.

Finally, all peptide reagents were lyophilized: min. 5 mg of each free peptide (4 competitor reagents), and min. 1 mg of each peptide-protein conjugate (4 standard reagents, at selected peptide:protein ratio).

Recombinant Proteins and SDS-PAGE Control (Histone, Erk)

8 recombinant proteins from different vendors were mutually selected as full protein alternatives to peptide standards (Table 6). 7 proteins (2× Histone H3, 5× Erk) were quality controlled by SDS-PAGE using BSA as a reference proteins.

TABLE 6
List of recombinant proteins.

| | | | | | | | | correction |
|---|---|---|---|---|---|---|---|---|
| | SDS | | | | ordering | | conc. | factor |
| # | PAGE | protein | species | supplier | no. | lot | [mg/ml] | (SDS-PAGE) |
| 1 | x | Histone H3 | calf | Roche | 11 034 758 001 | n.a. | 3.00 | 0.73 |
| 2 | x | Histone H3 | human | Upstate | 14-494 | 30 036 | 0.25 | 0.56 |
| 3 | x | ERK1 (p42) active (phospho) | human | Active Motif | 31 152 | 01 806 001 | 0.50 | 0.56 |
| 4 | | pRb | human | Active Motif | 31 128 | 07 904 001 | 0.50 | 1.00 |
| 5 | x | ERK1 (p44) | human | CST | 7416 | 1 | 0.10 | 0.50 |
| 6 | x | ERK1 (p44) inactive (GST tagged) | human | Invitrogen | PV 3312 | 32 403 A | 1.00 | 0.84 |
| 7 | x | ERK1 (p44) active (GST tagged) | human | Invitrogen | PV 3311 | 35 296 B | 0.40 | 0.89 |
| 8 | x | ERK2 (p42) | human | Biosource | PHO 0104 | P 092604 | 0.90 | 0.96 |

The purity of the proteins was good as evident from the single bands after the gel electrophoresis. However, signal instensities of the single bands showed large differences indicating different concentrations of the protein when compared to same amounts of BSA loaded as a reference. Obviously values of concentrations given in the data sheets of the vendors were not reliable. Therefore, the integral signal intensities of the protein bands were analyzed to estimate at best the right concentrations relative to co-loaded BSA. Resulting correction factors (see Table 6) were therefore considered in the sample preparation of all standard dilution series printed in the following.

Optimization of Assay and Print Conditions—First Standard Curves

In first experiments, assays were performed on different sets of arrays which were printed with standard curves of the different peptide-BSA reagents at different compositions and conditions, to examine their effects on subsequently tested immunoassay performance. The following conditions were examined:
Standard curves of peptide-BSA reagents with the different peptide:protein conjugate ratios (tested for HistoneH3-BSA, pRb-BSA and pErk-BSA)
Standard curves of peptide-BSA reagents printed in the absence and presence of additional protein matrix addition (BSA)

Standard curves were printed as 12 serial dilutions curves (2-fold dilutions), each dilution as duplicate spots (as described in Example 1: Material and Methods). Start concentrations of the different reagents for printing were adjusted to a uniform epitope concentration of 50 nM. In addition, positive and negative control lysates were co-printed into same arrays. The lysate samples were arrayed at a total protein concentration of 0.25 mg/ml. Immunoassays were performed at the antibody conditions indicated. Observed differences on assay performance were evaluated qualitatively and, based on these results and previous experience with these types of reagents, best print and assay conditions were selected.

Standard Curves of Peptide Reagents Containing Different Peptide:Protein Conjugate Ratios

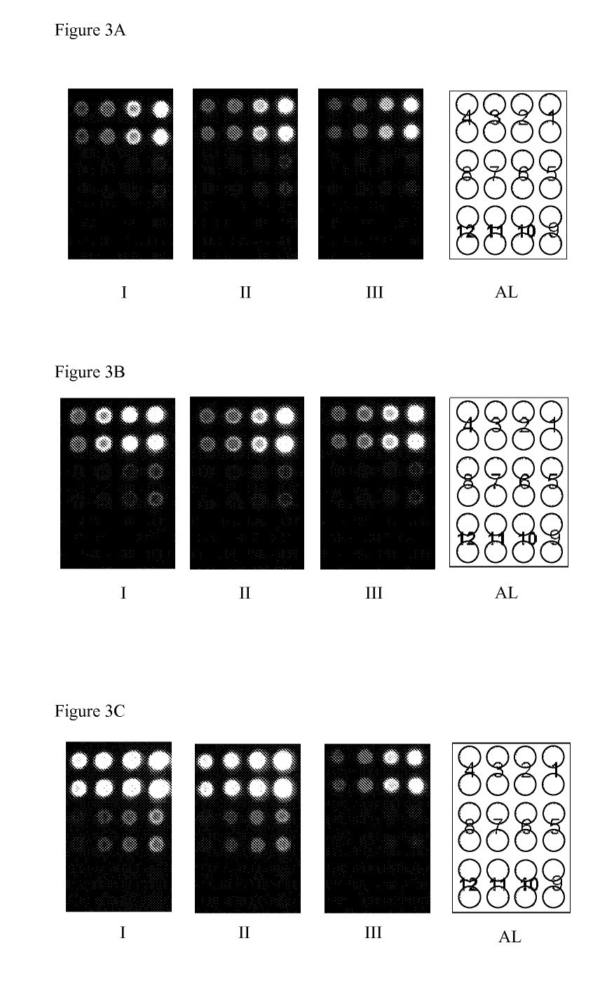

Generally, standard curves of printed peptide standard reagents at different peptide:protein ratios provided almost comparable signals in the assays. Assay images are depicted in FIG. 3. However, peptide standard reagents of the lowest peptide-protein conjugate ratios (much below 1) tended to show more donut-like spot morphologies, whereas reagents of the highest conjugate ratios tended to provide lower assay signal response (see especially for pErk1/2 assay). The latter trend may be interpreted as a lower (less than linear) binding accessibility of assay antibodies to immobilized peptide-BSA molecules containing more than one epitope sequence per BSA molecule (here typically 3-6 for the highest ratios). For the further experiments, we therefore selected the reagents of intermediate conjugation ratios: HistoneH3-BSA 2.7× (0.41 peptide:protein), pRb-BSA 1× (0.41 peptide:protein) and pErk-BSA 2.7× (0.89 peptide:protein).

Dynamic Range of Signals and Concentrations

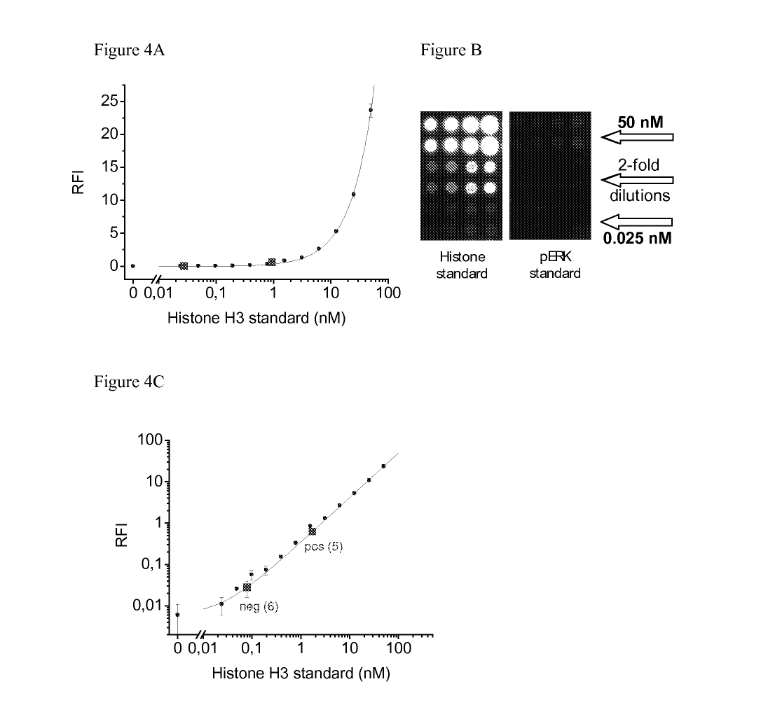

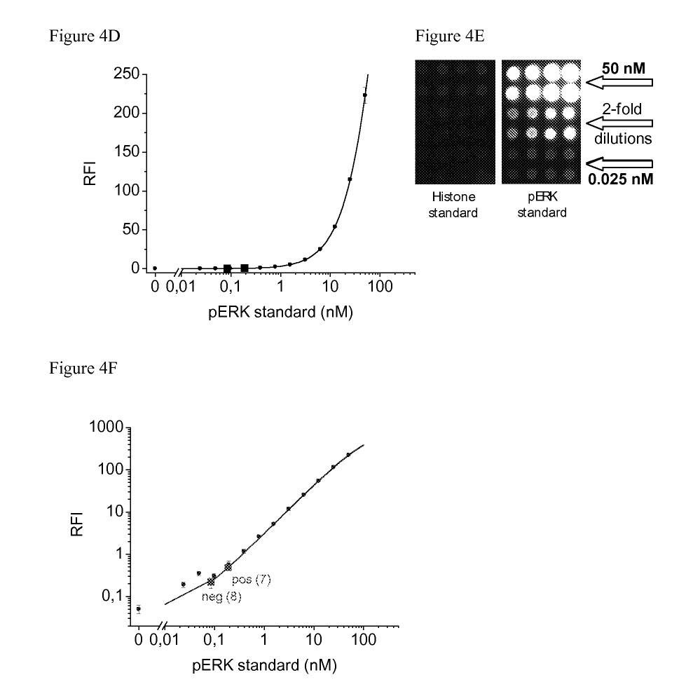

The assays on the printed standard curves demonstrated very prominent signals and a high dynamic range of signals which can be extracted from one and the same measurement in one image. FIG. 4 shows the analyzed quantitative signals representatively for the case of HistoneH3-BSA 2.7× and pErk-BSA 2.7×. The signals fitted perfectly to the low-end curve of a 1-site binding model (r²>0.99) with good linearity. The dynamic range of signals covered 4 orders of magnitude over 4 orders of concentration within one image (one exposure time). The dynamic range of the assay may be even further expanded by 1-2 orders (to our experience), since the reader allows image recordings at different exposure times and the use of different gray filters in addition.

The start concentrations of these first standard curves printed was chosen at 50 nM. In a signal comparison to the co-printed control lysate samples, it turned out that assays signals of the standard curves and hence the highest start concentration of standards were much higher than the intrinsic values (levels) of the respective control lysates, especially for the phophorylated protein analytes. Therefore start concentrations of standard curves had to be adjusted respectively. Also the signal differences of negative and positive control lysates, respectively expression levels, were very low, especially for the phosphorylated analytes pErk and pRb (obviously positive treatment of the prepared cell lines had been suboptimal). Therefore new control lysates were prepared and provided (see Table 1).

Standard Curves of Peptide-BSA Reagents in Absence/Presence of Matrix Protein Additions

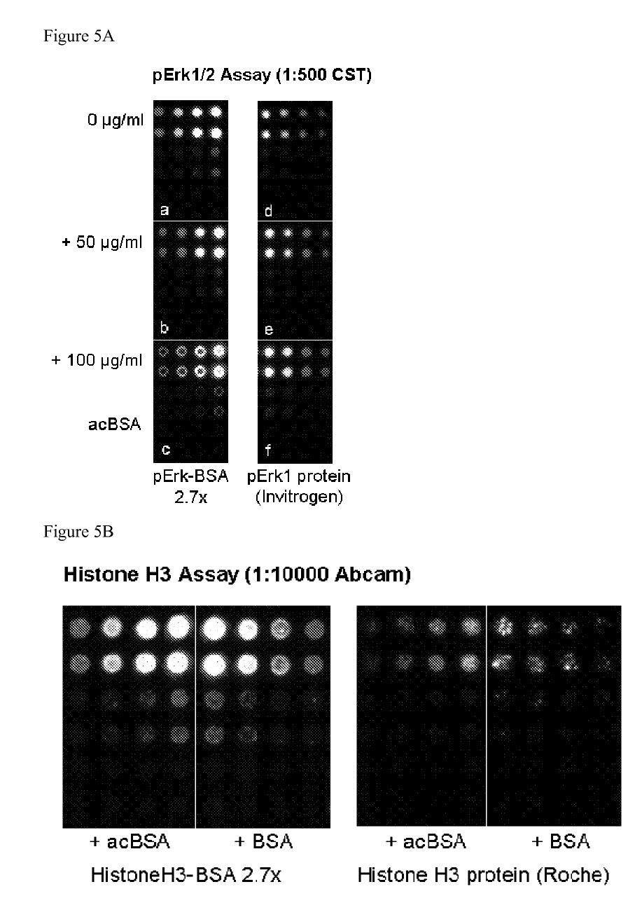

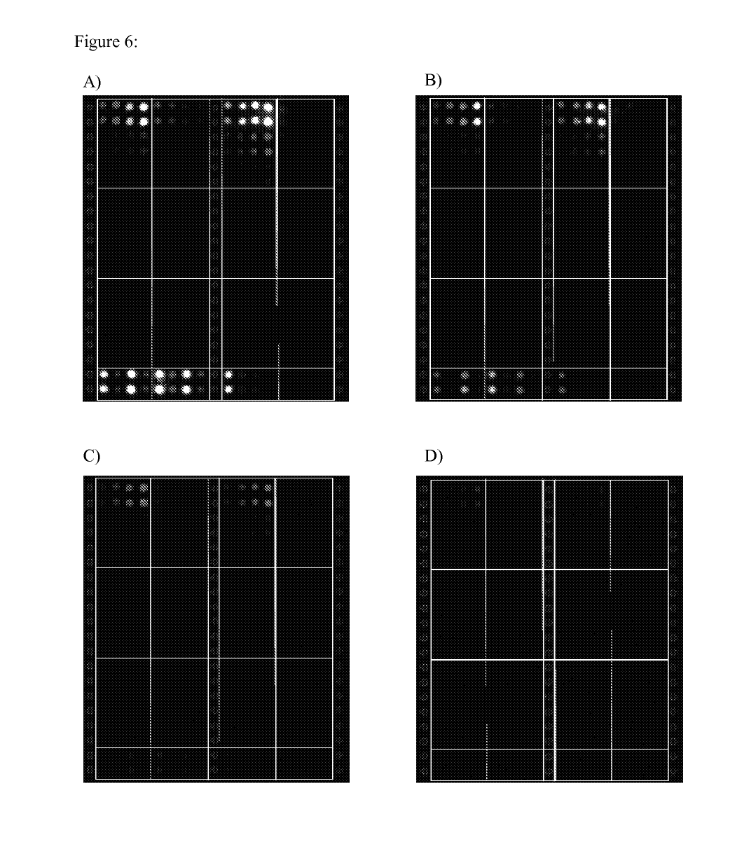

In another set of arrays, standard curves of peptide standard reagents and first recombinant proteins were printed at three different buffer conditions: (i) in the absence of any additional protein addition, and in the presence of (ii) 50 μg/ml an (iii) 100 μg/ml matrix protein (acetylated BSA=acBSA), as depicted in FIG. 5A. This was done to test the effect of matrix protein additions on spot morphology. Standard curve signals, generated in subsequently performed assays, showed that matrix protein addition generally led to a better and more homogeneous signal distribution as compared to no addition of matrix protein (as expected from other applications). This effect was observed for standard spots of peptide reagents as well as of recombinant proteins. At the same time, mean spot signals remained almost unchanged, indicating that protein additions mainly led to a reorganization of a constant number of standard molecules within the spot. Added matrix protein typically generated larger spot diameters which were better comparable to spot diameters of the lysate samples. This also made subsequent data analysis of standard and lysate sample spots more consistent. Additions of higher concentrations of acBSA matrix protein (100 μg/ml) led do donut shaped signal spot morphologies, for the peptide reagents. Another experiment was performed to examine the effect of the type of matrix protein: additions of non-modified and acetylated forms of BSA were directly compared in standard curves. The results are depicted in FIG. 5B (shown for Histone H3) and revealed that acBSA had the higher potency to generate homogeneous spot signals, especially for standard spots of recombinant proteins. Mean signals of spots were comparable. In a conclusion from our examinations so far, we have selected the best uniform condition for the printing of standard curves of peptide standard reagent as well as recombinant proteins (CSBL buffer plus 50 μg/ml acBSA).

Summary of best selected print and assay conditions for this study:

Uniform print condition selected for all standard curves:

Spotting buffer CSBL plus addition of 50 μg/ml acetylated BSA (acBSA)

Start concentrations selected for dilution series (peptide standards):

10 nM Histone H3

1 nM pRb

2.5 nM pERk1/2

5 nM ERk1/2

Assay conditions (antibody dilutions) selected:

1:10000 for Histone H3 assay

1:250 for pRb assay

1:500 for pErk1/2 assay

1:1000 for Erk1/2 assays (3 antibodies)

Signals of printed reference spots were adjusted to typically 15000 gray levels at 4 s image exposure time.

### Example 3

### Specificity of Calibration Reagents as Derived from Competition Experiments with Free Peptide in Solution

Arrays were printed with standard curves of the 4 peptide standard reagents (HistoneH3-BSA 2.7×, pRb-BSA 1×, pErk-BSA 2.7× and Erk1-BSA 2.7×) as well as all available recombinant proteins for comparison (12 standard curves with 12 point dilution curves). Control lysate sample controls (negative and positive controls, new delivery samples) were co-printed at a total protein concentration of 400 μg/ml and 250 μg/ml. Standards and control lysates were prepared in spotting buffer CSBL, standards with additions of 50 μg/ml acBSA. Start concentrations of the standard curve samples were adjusted to reach in minimum the assay signals of the positive control lysates. Array layout and conditions are summarized in Table 7.

TABLE 7
Array layout for competition experiments: Conditions of printed standard curves,
applied reagents and lysate controls. The numbers in the first column refer to the array fields or
the position in the control fields shown in FIG. 1B.

| Array | | | | | Start | |
|---|---|---|---|---|---|---|
| field | Type | Standard reagent | | Supplier | conc. | |
| 1 | Standard curve (12x/2x) | Histone H3-BSA (2.7x) | | NMI peptide conjugate | 10 | nM |
| 2 | Standard curve (12x/2x) | Histone H3 | | Roche | 10 | nM |
| 3 | Standard curve (12x/2x) | Histone H3 | | Upstate | 28 | nM |
| 4 | Standard curve (12x/2x) | Erk1 | | Invitrogen | 7.5 | nM |
| 5 | Standard curve (12x/2x) | pErk1/2-BSA (2.7x) | | NMI peptide conjugate | 2.5 | nM |
| 6 | Standard curve (12x/2x) | Erk1-BSA (2.7x) | | NMI peptide conjugate | 5 | nM |
| 7 | Standard curve (12x/2x) | Erk1 phosphorylated | | Active Motif | 1.5 | nM |
| 8 | Standard curve (12x/2x) | Erk1 phosphorylated | | Invitrogen | 6 | nM |
| 9 | Standard curve (12x/2x) | pRb-BSA (1.0x) | | NMI peptide conjugate | 1 | nM |
| 10 | Standard curve (12x/2x) | pRb (Rb phosphorylated?) | | Active Motif | 1 | nM |
| 11 | Standard curve (12x/2x) | Erk2 | | Biosource | 5.4 | nM |
| 12 | Standard curve (12x/2x) | Erk1 | | CST | 2.2 | nM |
| | Controls | | | | total protein conc. | |
| 1 | Lysate#7new pErk(+) | | | | 400 | μg/ml |
| 2 | Lysate#7new pErk(+) | | | | 250 | μg/ml |
| 3 | Lysate#8new pErk(−) | | | | 400 | μg/ml |
| 4 | Lysate#8new pErk(−) | | | | 250 | μg/ml |
| 5 | Lysate#9new pRb(+) | | | | 400 | μg/ml |
| 6 | Lysate#9new pRb(+) | | | | 250 | μg/ml |
| 7 | Lysate#10new pRb(−) | | {close oversize brace} | Roche | 400 | μg/ml |
| 8 | Lysate#10new pRb(−) | | | | 250 | μg/ml |
| 9 | Lysate #5 Histone H3 (+) | | | | 250 | μg/ml |
| 10 | Lysate #5 Histone H3 (+) | | | | 150 | μg/ml |
| 11 | Lysate #6 Histone H3 (−) | | | | 150 | μg/ml |
| 12 | Lysate #6 Histone H3 (−) | | | | 90 | μg/ml |
| 13 | Spotting Buffer CSBL | | | | 0 | |
| 14 | Spotting Buffer CSBL | | | | 0 | |
| 15 | Lysate buffer CLB1:CSBL(1:10) | | | | 0 | |
| 16 | Lysate buffer CLB1:CSBL(1:10) | | | | 0 | |

Assay were performed on the arrays for each of the four protein analytes in the absence (normal assay) and presence of increasing concentrations of corresponding free peptide, which was pre-mixed with the respective antibody solution before incubation on the arrays (competition assays). Typically three different concentrations of free peptide (1000 nM, 100 nM and 10nM, if not otherwise indicated) were tested for their efficiency to complex with respective antibody to suppress the formation of specific antibody-protein analyte complexes on the array spots. In addition, competition assay were performed with antibody solutions which were pre-incubated with corresponding recombinant proteins, to compare their competition efficiency and specificity with that of the free peptide reagents. Blank assays (in absence of primary antibody) were performed as additional controls but their signals were negligibly low and therefore were not considered in the quantitative data analysis.

Tables 8 to 11 summarize the quantitative results in terms of maximum standard curve signals.

The results of the Erk1/2 assays nicely demonstrated that not only the Erk1/2 antibody form Biosource, but also the two additionally chosen CST antibodies (#4695 rb monoclonal and #9102 rb polyclonal) specifically recognized only the Erk1-BSA standard spots (and at comparable signal intensities), but not the pErk-BSA standard spots. This implies that all three antibodies from the three different vendors used in this project obviously were raised against a very similar peptide motif at the C-terminal end of the protein. This was further corroborated by an additional earlier competition experiment (add-on experiment), which was performed with the Erk1/2 antibody form CST (#9102) in the presence of a free epitope peptide which represented the amino acid sequence of the Erk1/2 phosphorylation site (as used in this project) but was not phosphorylated (de-phospho peptide, available at NMI). In the competition assay, performed under otherwise comparable conditions as was shown before, this de-phospho peptide was not able to suppress the specific signals of the standard and lysate spots observed in the respective Erk1/2 normal assay (data not shown). It is therefore shown that the Erk antibodies purchased from CST use different protein epitope sequences to differentiate between the total and phosphorylated forms of the Erk1/2 protein.

TABLE 8
Table of signal values (maxima) of standard curve signals of all array fields for Histone H3 assay (1:10′000).

| | | Maximum standard curve signals (RFI) | | | | | | | | | | | | | |
|---|---|---|---|---|---|---|---|---|---|---|---|---|---|---|---|
| | | | | Competition assay | | | | | | | | | | | |
| | | | | +pep- | | +pep- | | +pep- | | +pep- | | +pro- | | +pro- | |
| Array | Type | Normal assay | | tide | | tide | | tide | | tide | | tein | | tein | |
| field | standard reagent | signal | std | 10000 nM | std | 1000 nM | std | 100 nM | std | 10 nM | std | 100 nM | std | 10 nM | std |
| 1 | Histone | 5.38 | 0.322 | 0.19 | 0.010 | 1.41 | 0.127 | 3.33 | 0.080 | 4.28 | 0.088 | 1.68 | 0.228 | 3.93 | 0.267 |
| | H3-BSA 2.7x | | | | | | | | | | | | | | |
| 2 | Histone H3 Roche | 0.43 | 0.015 | <0.01 | 0.001 | 0.04 | 0.004 | 0.20 | 0.004 | 0.30 | 0.027 | 2.56 | 0.214 | 2.50 | 0.074 |
| 3 | Histone H3 Upstate | 11.18 | 0.195 | 0.07 | 0.007 | 1.13 | 0.031 | 4.45 | 0.155 | 7.04 | 0.115 | 2.09 | 0.062 | 3.16 | 0.269 |
| 4 | Erk1 Invitrogen | 0.02 | 0.000 | <0.01 | 0.001 | 0.01 | 0.016 | 0.02 | 0.000 | <0.01 | 0.042 | 1.32 | 0.528 | 2.54 | 0.359 |
| 5 | pErk-BSA 2.7x | <0.01 | 0.000 | 0.02 | 0.003 | <0.01 | 0.000 | <0.01 | 0.001 | <0.01 | 0.000 | 1.81 | 0.201 | 2.57 | 0.164 |
| 6 | Erk1-BSA 2.7x | 0.01 | 0.002 | 0.01 | 0.002 | <0.01 | 0.000 | 0.01 | 0.003 | <0.01 | 0.001 | 1.42 | 0.031 | 2.80 | 0.179 |
| 7 | pErk Active Motif | <0.01 | 0.002 | <0.01 | 0.001 | <0.01 | 0.001 | <0.01 | 0.001 | <0.01 | 0.001 | 1.87 | 0.131 | 2.40 | 0.040 |
| 8 | pErk1 Invitrogen | <0.01 | 0.002 | <0.01 | 0.001 | <0.01 | 0.000 | <0.01 | 0.000 | <0.01 | 0.002 | 1.37 | 0.062 | 2.20 | 0.227 |
| 9 | pRb-BSA 1x | <0.01 | 0.002 | <0.01 | 0.000 | <0.01 | 0.001 | <0.01 | 0.001 | <0.01 | 0.000 | 2.46 | 0.149 | 2.57 | 0.603 |
| 10 | pRb Active Motif | <0.01 | 0.001 | <0.01 | 0.001 | <0.01 | 0.002 | <0.01 | 0.000 | <0.01 | 0.002 | 2.28 | 0.517 | 2.77 | 0.060 |
| 11 | Erk2 Biosource | <0.01 | 0.002 | <0.01 | 0.001 | <0.01 | 0.003 | <0.01 | 0.003 | <0.01 | 0.002 | 1.56 | 0.103 | 2.30 | 0.041 |
| 12 | Erk1 CST | <0.01 | 0.000 | <0.01 | 0.000 | <0.01 | 0.001 | <0.01 | 0.001 | <0.01 | 0.003 | 1.80 | 0.163 | 2.19 | 0.025 |

Signals of specific standard curves are underlined.

TABLE 9
Table of signal values (maxima) of standard curve signals of all array fields for pRb assay (1:250).

| | | Maximum standard curve signals (RFI) | | | | | | | |
|---|---|---|---|---|---|---|---|---|---|
| | | | | Competition assay | | | | | |
| Array | Type | Normal assay | | +peptide | | +peptide | | +peptide | |
| field | standard reagent | signal | std | 1000 nM | std | 100 nM | std | 10 nM | std |
| 1 | Histone H3-BSA 2.7x | 0.03 | 0.004 | 0.01 | 0.000 | 0.02 | 0.004 | 0.02 | 0.004 |
| 2 | Histone H3 Roche | <0.01 | 0.000 | <0.01 | 0.000 | <0.01 | 0.000 | <0.01 | 0.001 |
| 3 | Histone H3 Upstate | 0.01 | 0.000 | <0.01 | 0.000 | <0.01 | 0.000 | <0.01 | 0.000 |
| 4 | Erk1 Invitrogen | <0.01 | 0.001 | <0.01 | 0.001 | <0.01 | 0.000 | <0.01 | 0.000 |
| 5 | pErk-BSA 2.7x | <0.01 | 0.001 | <0.01 | 0.001 | <0.01 | 0.001 | 0.01 | 0.003 |
| 6 | Erk1-BSA 2.7x | 0.01 | 0.001 | <0.01 | 0.001 | <0.01 | 0.000 | <0.01 | 0.000 |
| 7 | pErk Active Motif | <0.01 | 0.000 | <0.01 | 0.000 | <0.01 | 0.000 | <0.01 | 0.000 |
| 8 | pErk1 Invitrogen | <0.01 | 0.000 | <0.01 | 0.000 | <0.01 | 0.000 | <0.01 | 0.001 |
| 9 | pRb-BSA 1x | 0.47 | 0.019 | <0.01 | 0.001 | <0.01 | 0.003 | 0.07 | 0.003 |
| 10 | pRb Active Motif | 0.04 | 0.002 | <0.01 | 0.000 | <0.01 | 0.000 | <0.01 | 0.001 |
| 11 | Erk2 Biosource | <0.01 | 0.000 | <0.01 | 0.000 | <0.01 | 0.000 | <0.01 | 0.001 |
| 12 | Erk1 CST | <0.01 | 0.000 | <0.01 | 0.000 | <0.01 | 0.000 | <0.01 | 0.000 |

Maximum signals of specific standard curves are indicated in bold.

TABLE 10
Table of signal values (maxima) of standard curve signals
of all array fields for pErk1/2 assay (1:500).

| | | Maximum standard curve signals (RFI) | | | | | | | |
|---|---|---|---|---|---|---|---|---|---|
| | | | | Competition assay | | | | | |
| Array | Type | Normal assay | | +peptide | | +peptide | | +peptide | |
| field | standard reagent | signal | std | 1000 nM | std | 100 nM | std | 10 nM | std |
| 1 | Histone H3-BSA 2.7x | 0.11 | 0.012 | 0.10 | 0.004 | 0.10 | 0.003 | 0.10 | 0.001 |
| 2 | Histone H3 Roche | 0.01 | 0.000 | 0.01 | 0.003 | 0.01 | 0.000 | 0.01 | 0.002 |
| 3 | Histone H3 Upstate | 0.01 | 0.001 | 0.01 | 0.003 | <0.01 | 0.000 | 0.01 | 0.001 |
| 4 | Erk1 Invitrogen | 0.23 | 0.007 | 0.01 | 0.001 | 0.01 | 0.002 | 0.01 | 0.001 |
| 5 | pErk-BSA 2.7x | 3.47 | 0.092 | 0.10 | 0.018 | 0.45 | 0.024 | 1.05 | 0.062 |
| 6 | Erk1-BSA 2.7x | 0.02 | 0.001 | 0.02 | 0.004 | 0.02 | 0.007 | 0.01 | 0.002 |
| 7 | pErk Active Motif | 0.19 | 0.019 | 0.01 | 0.006 | 0.01 | 0.002 | 0.01 | 0.001 |
| 8 | pErk1 Invitrogen | 1.66 | 0.050 | 0.01 | 0.001 | 0.01 | 0.004 | 0.05 | 0.007 |
| 9 | pRb-BSA 1x | 0.01 | 0.001 | 0.01 | 0.003 | 0.01 | 0.000 | 0.01 | 0.005 |
| 10 | pRb Active Motif | 0.01 | 0.000 | 0.01 | 0.004 | 0.01 | 0.001 | 0.01 | 0.001 |
| 11 | Erk2 Biosource | 0.01 | 0.000 | 0.01 | 0.000 | 0.01 | 0.003 | 0.01 | 0.001 |
| 12 | Erk1 CST | 0.04 | 0.002 | <0.01 | 0.000 | <0.01 | 0.002 | 0.01 | 0.002 |

Maximum signals of specific standard curves are indicated in bold.

TABLE 11
Table of signal values (maxima) of standard curve signals of all array fields for Erk1/2
assay conducted with 3 different antibodies at 1:1000 dilutions: (top) Biosource 44-654G,
(middle) CST #4695 rb monoclonal, and (bottom) CST #9102 rb polyclonal antibody.

| | | Maximum standard curve signals (RFI) | | | | | | | |
|---|---|---|---|---|---|---|---|---|---|
| | | | | Competition assay | | | | | |
| Array | Type | Normal assay | | +peptide | | +protein | | +protein | |
| field | standard reagent | signal | std | 1000 nM | std | 100 nM | std | 10 nM | std |
| Biosource 44-654G | | | | | | | | | |
| rb polyclon | | | | | | | | | |
| 1 | Histone H3-BSA 2.7x | 0.04 | 0.000 | 0.02 | 0.001 | 0.22 | 0.019 | 0.13 | 0.013 |
| 2 | Histone H3 Roche | 0.02 | 0.003 | 0.01 | 0.001 | 0.15 | 0.015 | 0.07 | 0.004 |
| 3 | Histone H3 Upstate | <0.01 | 0.006 | 0.01 | 0.003 | 0.16 | 0.014 | 0.09 | 0.003 |
| 4 | Erk1 Invitrogen | 10.91 | 0.409 | 0.02 | 0.001 | 0.38 | 0.014 | 5.17 | 0.056 |
| 5 | pErk-BSA 2.7x | <0.01 | 0.037 | 0.01 | 0.000 | 0.14 | 0.022 | 0.08 | 0.031 |
| 6 | Erk1-BSA 2.7x | 8.24 | 0.356 | 0.14 | 0.018 | 5.69 | 0.327 | 7.88 | 0.210 |
| 7 | pErk Active Motif | 1.17 | 0.129 | 0.01 | 0.000 | 0.15 | 0.003 | 0.31 | 0.007 |
| 8 | pErk1 Invitrogen | 10.43 | 0.372 | 0.02 | 0.003 | 0.32 | 0.026 | 3.88 | 0.010 |
| 9 | pRb-BSA 1x | 0.03 | 0.003 | 0.01 | 0.003 | 0.16 | 0.001 | 0.10 | 0.010 |
| 10 | pRb Active Motif | 0.02 | 0.004 | 0.01 | 0.002 | 0.17 | 0.001 | 0.11 | 0.006 |
| 11 | Erk2 Biosource | 3.57 | 0.085 | 0.01 | 0.001 | 0.18 | 0.005 | 0.90 | 0.095 |
| 12 | Erk1 CST | 0.28 | 0.017 | <0.01 | 0.001 | 0.16 | 0.004 | 0.15 | 0.002 |
| CST #4695 | | | | | | | | | |
| rb monoclonal ab | | | | | | | | | |
| 1 | Histone H3-BSA 2.7x | 0.01 | 0.001 | 0.01 | 0.001 | 0.12 | 0.006 | no assay | — |
| 2 | Histone H3 Roche | 0.01 | 0.001 | <0.01 | 0.001 | 0.08 | 0.001 | no assay | — |
| 3 | Histone H3 Upstate | 0.01 | 0.002 | <0.01 | 0.001 | 0.10 | 0.007 | no assay | — |
| 4 | Erk1 Invitrogen | 7.12 | 0.152 | 0.04 | 0.001 | 0.59 | 0.006 | no assay | — |
| 5 | pErk-BSA 2.7x | <0.01 | 0.007 | 0.01 | 0.002 | 0.09 | 0.016 | no assay | — |
| 6 | Erk1-BSA 2.7x | 2.77 | 0.159 | 0.01 | 0.002 | 0.29 | 0.015 | no assay | — |
| 7 | pErk Active Motif | 0.22 | 0.052 | <0.01 | 0.000 | 0.11 | 0.001 | no assay | — |
| 8 | pErk1 Invitrogen | 5.17 | 0.373 | 0.02 | 0.000 | 0.39 | 0.003 | no assay | — |
| 9 | pRb-BSA 1x | 0.01 | 0.002 | 0.01 | 0.000 | 0.10 | 0.002 | no assay | — |
| 10 | pRb Active Motif | 0.01 | 0.001 | <0.01 | 0.002 | 0.09 | 0.004 | no assay | — |
| 11 | Erk2 Biosource | 1.51 | 0.072 | 0.01 | 0.001 | 0.16 | 0.002 | no assay | — |
| 12 | Erk1 CST | 0.04 | 0.003 | <0.01 | 0.002 | 0.10 | 0.001 | no assay | — |
| CST #9102 | | | | | | | | | |
| rb polyclonal ab | | | | | | | | | |
| 1 | Histone H3-BSA 2.7x | 0.08 | 0.007 | 0.07 | 0.006 | 0.10 | 0.008 | no assay | — |
| 2 | Histone H3 Roche | <0.01 | 0.000 | <0.01 | 0.001 | 0.03 | 0.005 | no assay | — |
| 3 | Histone H3 Upstate | <0.01 | 0.008 | <0.01 | 0.002 | 0.03 | 0.001 | no assay | — |
| 4 | Erk1 Invitrogen | 8.96 | 0.047 | 7.28 | 0.221 | 0.19 | 0.000 | no assay | — |
| 5 | pErk-BSA 2.7x | <0.01 | 0.006 | 0.01 | 0.002 | 0.04 | 0.004 | no assay | — |
| 6 | Erk1-BSA 2.7x | 2.44 | 0.002 | 0.06 | 0.000 | 0.12 | 0.004 | no assay | — |
| 7 | pErk Active Motif | 0.36 | 0.041 | 0.23 | 0.007 | 0.03 | 0.004 | no assay | — |
| 8 | pErk1 Invitrogen | 7.20 | 0.261 | 4.65 | 0.049 | 0.11 | 0.001 | no assay | — |
| 9 | pRb-BSA 1x | 0.01 | 0.001 | 0.01 | 0.001 | 0.04 | 0.007 | no assay | — |
| 10 | pRb Active Motif | <0.01 | 0.002 | <0.01 | 0.000 | 0.04 | 0.004 | no assay | — |
| 11 | Erk2 Biosource | 1.51 | 0.222 | 0.91 | 0.034 | 0.07 | 0.003 | no assay | — |
| 12 | Erk1 CST | 0.05 | 0.000 | 0.03 | 0.003 | 0.03 | 0.001 | no assay | — |

Maximum signals of specific standard curves are indicated in bold.

### Example 4

### Sensitivity of Calibration Reagent—Limits-of-Detection (LOD)

All assays were performed on arrays of the same layout as shown in FIG. 1B and table 7. Arrays comprised the standard curves of all 4 peptide standard reagents (HistoneH3-BSA 2.7×, pRb-BSA 1×, pErk-BSA 2.7× and Erk1-BSA 2.7×) as well as all available recombinant proteins for comparison (12 standard curves with 12 point dilution curves). Highest concentrations of the peptide standard curves were adjusted to 10 nM for Histone H3 standards, 1 nM for pRb standards, 2.5 nM for pErk standards and 5 nM for Erk standards. These start concentrations were chosen according to the highest endogenous signals generated by the positive control lysates. Concentrations of protein standards were prepared accordingly and adjusted applying the SDS-PAGE correction factors. Control lysate samples (negative and positive controls, including new delivery) were co-printed at a total protein concentration of 400 μg/ml (for stocks available with ≧4 mg/ml protein concentration) and 250 μg/ml. Standards and control lysates were prepared in spotting buffer CSBL, standards with additions of 50 μg/ml acBSA.

Assays were performed for each of the four protein analytes in the absence (normal assay) and presence of free peptide at the highest concentration effective for complete competition (competition assays). Each condition (normal assay, competition assay) was measured in duplicate assays (two arrays per condition). Blank assays (in absence of primary antibody) were additionally measured as a control. All array images were analyzed quantitatively. For each assay, standard signal curves for each of the 12 array fields of each array were generated by fitting a one-site binding model to the data points extracted from each of the printed 12-point dilution series. Limits-of-detection (LOD) were determined from the fit curve as back-calculated concentrations which corresponded to the mean signals at blank levels (four lowest data points) plus 3-fold respective standard deviations.

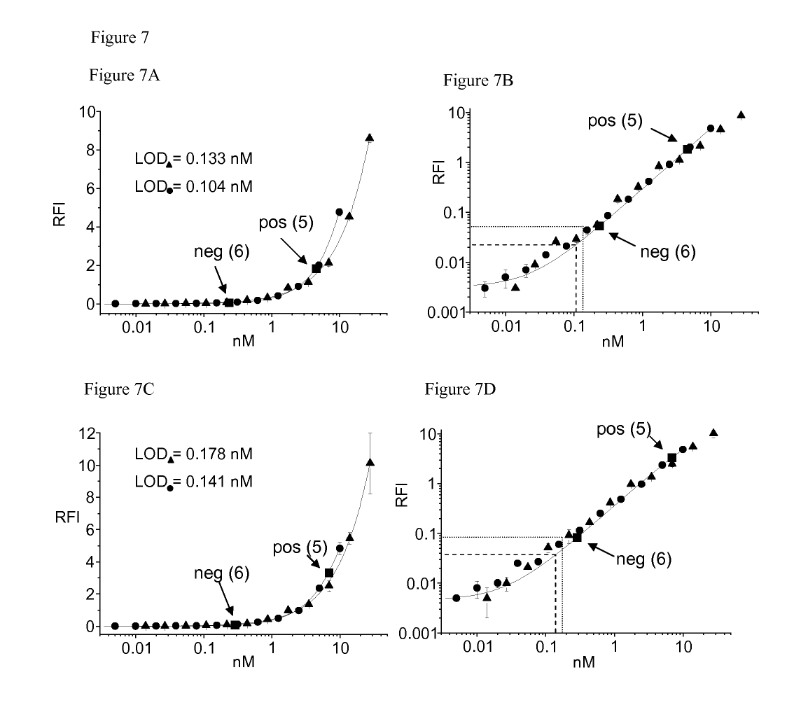

Generated standard curves of the normal and competition assays (data points and fitting curves, as well as back-calculated LOD values) for the duplicate assays are shown in the FIGS. 7 to 11. LODs are given in the graphs for each standard curve. Good quality of fit curves were achieved with correlation coefficients of r²>0.99.

Abcam antibody specifically bound to HistoneH3-BSA peptide standard and the Abcam antibody specifically bound also to Histone H3 recombinant proteins, most prominently to the human protein from Upstate. Signal intensities of standard curves of peptide standard and recombinant protein (Upstate) were well comparable. Reproducibilities of the two assays were very good. Signal CVs were typically about 12% for the peptide standards and about 13% for Histone H3 protein (Upsate). The mean LODs were 0.123±0.019 nM for peptide standard, and 0.156±0.023 nM for recombinant protein (Upstate). LOD values were well reproducible for the duplicate assays and comparable for peptide standard and protein (FIG. 7).

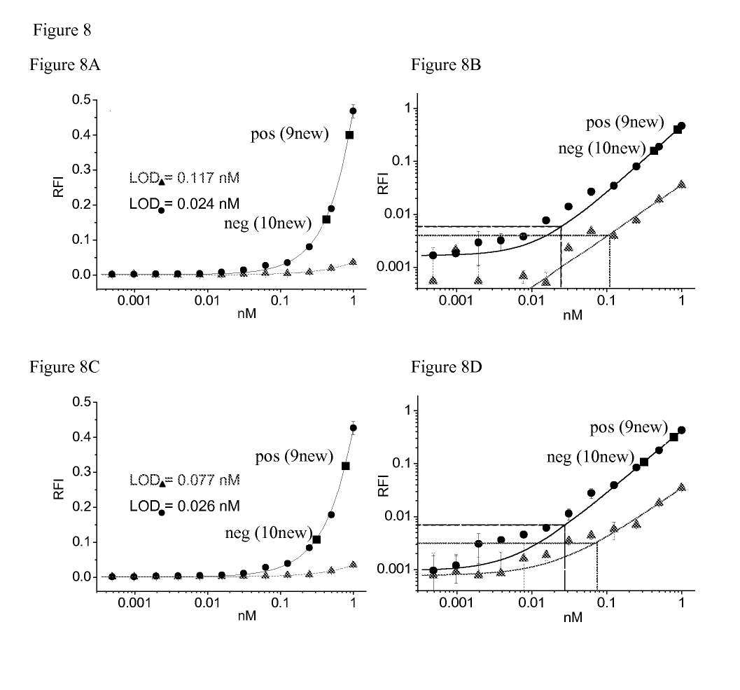

The CST antibody specifically bbound to pRb-BSA peptide standard and the CST antibody specifically bound also to pRb recombinant protein from Active Motif. However the signal intensities of the protein standard curves were clearly lower and reach only about 10% .of the peptide standard curves. We presume that the protein is not or only partly phosphorylated (note: pRb and Rb annotation in public data banks is obviously used in parallel for the same protein and it was not clear to us whether pRb used here indicated the phosphorylated protein). Reproducibilities of the two assays were very good. Signal CVs were typically about 7% for the peptide standards and slightly higher at about 12% for pRb protein. LOD values were well reproducible for the duplicate assays. The mean LODs were 0.025±0.001 nM for peptide standard, and 0.097±0.020 nM for recombinant protein (FIG. 8.

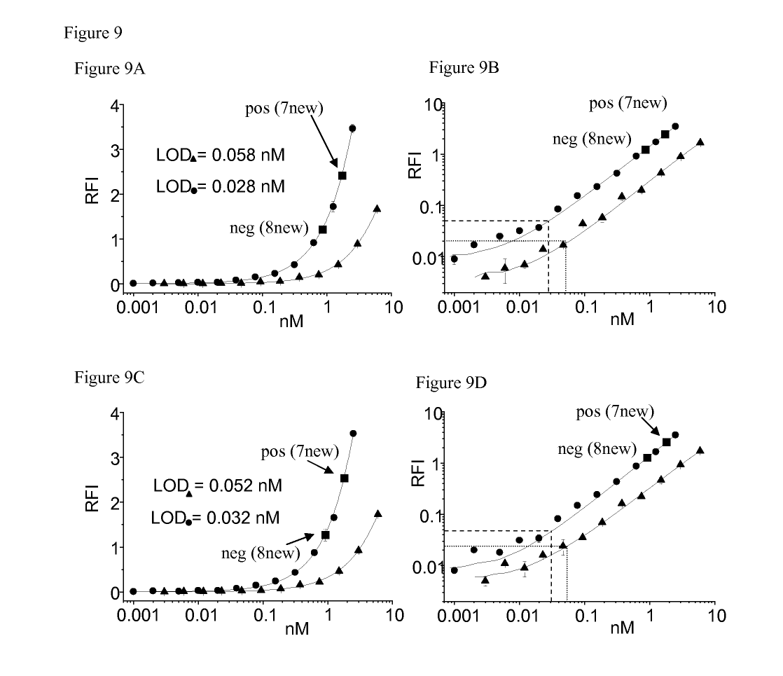

The CST antibody specifically bound only to pErk1/2-BSA peptide standard, and not to Erk1-BSA standard. The CST antibody specifically bound also prominently to the pErk1 recombinant protein (Invitrogen) and reaches signal intensities of about 25% of the respective pErk1/2-BSA peptide standard signals. The CST antibody bound to a minor degree also to pErk from Active Motif (about 12%) ≧Erk1 from CST (about 11%) ≧Erk1 protein from Invitrogen (about 3%). Signals are given relative to the signal of pErk1 protein (Invitrogen) in %. Reproducibilities of the two assays were very good. Signal CVs were typically about 2% for the peptide standard, and slightly higher at about 6% for the pErk1 protein (Invitrogen). LOD values were well reproducible for the duplicate assays. The mean LODs were 0.030±0.002 nM for peptide standard, and 0.055±0.003 nM for pErk1 protein (Invitrogen) (FIG. 9).

Good quality of fit curves achieved with correlation coefficients of r²>0.99

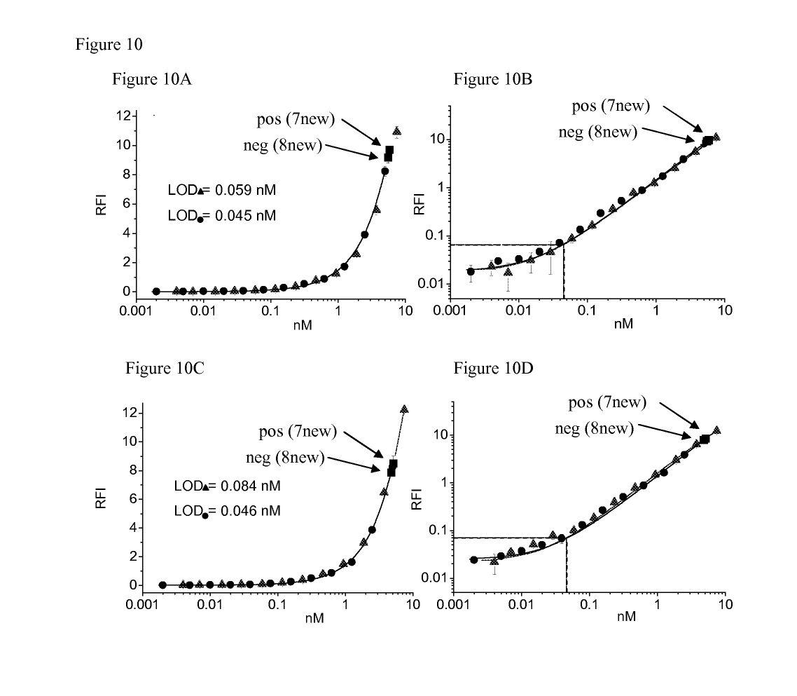

Biosource antibody specifically bound only to Erk1-BSA peptide standard, not to pErk1-BSA standard. Biosource antibody specifically bound to Erk1 and pErk1 recombinant proteins (most prominently among the different proteins available), and generated well comparable signal intensities for these proteins and Erk1-BSA peptide standards. Biosource antibody bound to a lower degree also to Erk2 protein (Biosource)>pErk (Active Motif)>Erk1 (CST). Reproducibilities of the two assays were very good. Signal CVs were typically about 3% for the peptide standard, and slightly higher about 8% for Erk1 protein and about 5%for pErk1 protein. LOD values were well reproducible for the duplicate assays. The mean LODs were 0.046±0.001 nM for peptide standard, and 0.072±0.013 nM for recombinant Erk1 protein (Invitrogen), and 0.044±0.004 nM for recombinant pErk1protein (Invitrogen) (FIGS. 10 and 11).

Good quality of fit curves achieved with correlation coefficients of r²>0.99

### Example 5

### Standard Curves Spiked into Lysates (5 and 10 replicate spots)

Determination of Absolute Protein Analyte Concentrations

Assays were performed on arrays of the layout shown in the following FIG. 12. Arrays comprised the dilution series of the 3 peptide standard reagents HistoneH3-BSA 2.7×, pRb-BSA 1× and pErk-BSA 2.7×. Dilution series were printed as 8 point series with 2-fold dilutions. Two types of dilution series were printed for each peptide reagent: one series was printed in spotting buffer (CSBL plus 50 μg/ml acBSA) similar to example 4, applying the same start concentrations as used for example 4. The other series was printed as a 7-point dilution series spiked into lysates which were negative for the respective protein. The applied total protein concentration of the lysates in the spiked dilution series was kept constant at 150 μg/ml. The highest start concentration spiked into the lysate was chosen as half of the start concentration of the respective series in buffer. As last sample of each spike-in series, the pure negative lysate (in absence of any spike concentration) was printed as a blank control.

TABLE 11
Conditions of printed standard dilution series, applied reagents
and spiked lysates are given in Table 1. Assay layout is shown
in FIG. 12.

| Array | Type of curve | | Start |
|---|---|---|---|
| field | (#points/#replicate spots) | Standard reagent | conc. |
| 1 | Standard dilution series | pErk-BSA (2.7x) | 2.5 nM |
| | (8x/10x) in buffer | | |
| 2 | Dilution series (8x/10x) | pErk-BSA (2.7x) | 1.25 nM |
| | spiked into pErk(−) lysate 13 | | |
| 3 | Standard dilution series | HistoneH3-BSA (2.7x) | 10 M |
| | (8x/5x) in buffer | | |
| 4 | Dilution series (8x/5x) | HistoneH3-BSA (2.7x) | 5 nM |
| | spiked into HisH3(−) lysate 6 | | |
| 5 | Standard dilution series | pRb-BSA (1.0x) | 1 nM |
| | (8x/5x) in buffer | | |
| 6 | Dilution series (8x/5x) | pRb-BSA (1.0x) | 0.5 nM |
| | spiked into pRb(−) lysate 12 | | |

Duplicate assays (on 2 arrays) were performed for each of the four protein analytes. Blank assays (in absence of primary antibody) were additionally measured as a control. All array images were analyzed quantitatively. For each assay, standard signal curves for each of the 6 array fields of each array were generated by fitting a one-site binding model to the data points extracted from each of the printed 8-point dilution series. Data points were averaged for the maximum number of replicate spots available in each series (N=5 or N=10). For a comparison, mean signals were also calculated for duplicate spots (center rows of each field). Limits-of-detection (LODs) were determined from the fit curves as described before. In addition, signals of spike-in series were corrected for the endogenous (blank) signals and corrected signal were projected into the standard curves in buffer.

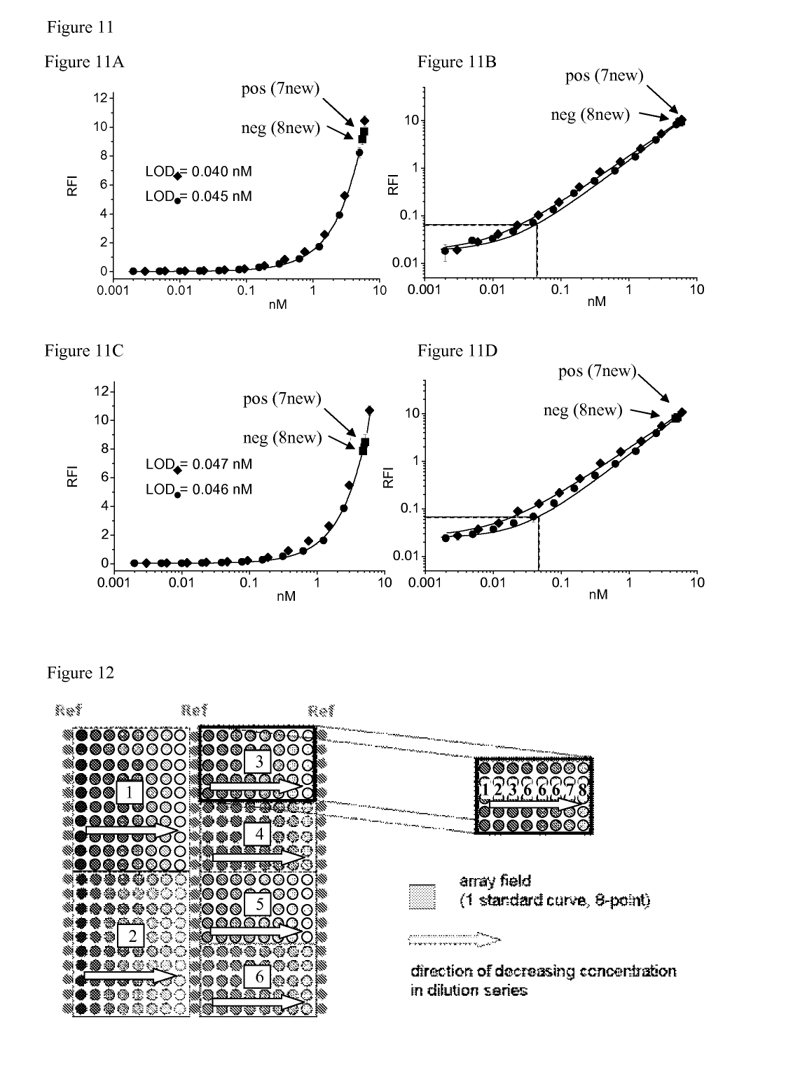

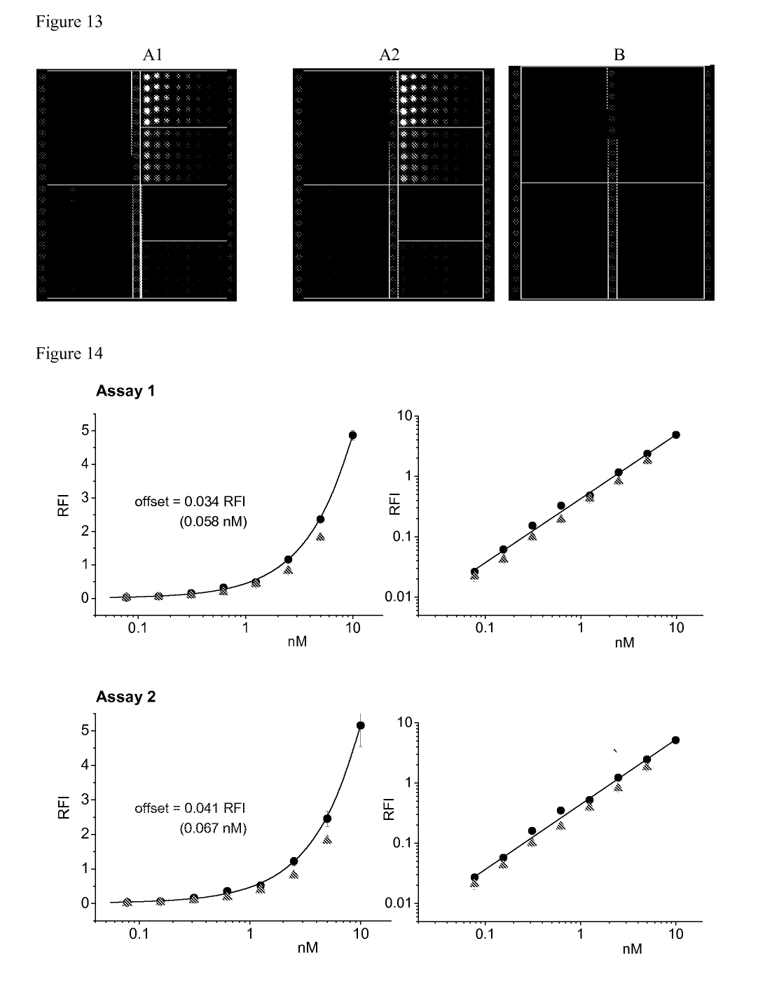

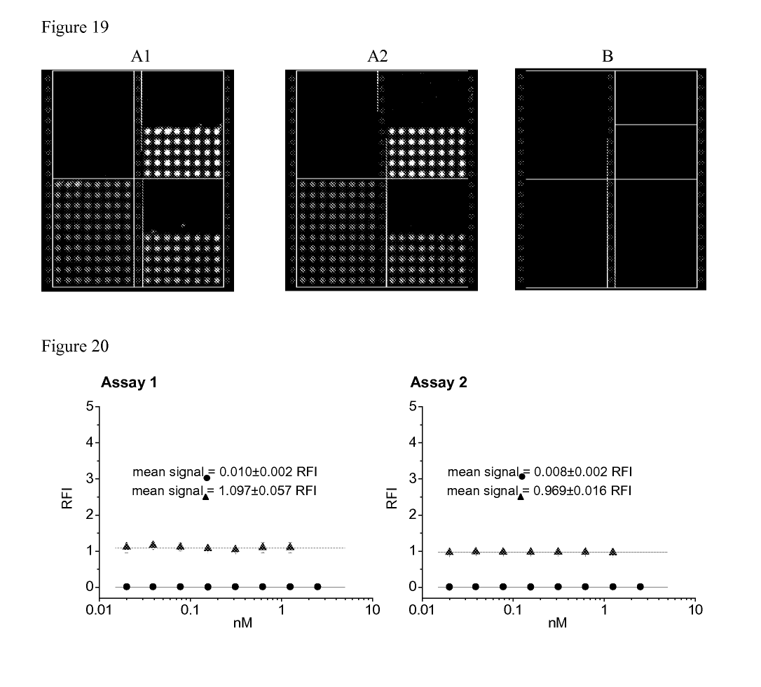

Results are shown in FIGS. 13 to 20.
Histone H3 peptide: The assays revealed the specific signal response for dilutions curves of the Histone H3 peptide standard. Blank assay showed zero response. Signals of lysate spiked with Histone H3 followed the signals of the standard curve in buffer at comparable, but slightly lower offset intensities (after subtraction of endogenous Histone H3 signal level of the pure lysate). Reproducibilities of the two assays were very good. Endogenous concentration of Histone H3 protein in pure lysate was determined by back-calculation of the blank signals of lysate from standard curve fits. The mean concentration was 0.063±0.005 nM. Other lysate spots showed marginally low signals (FIGS. 13 and 14). Good quality of fit curves achieved with correlation coefficients of r2 >0.99

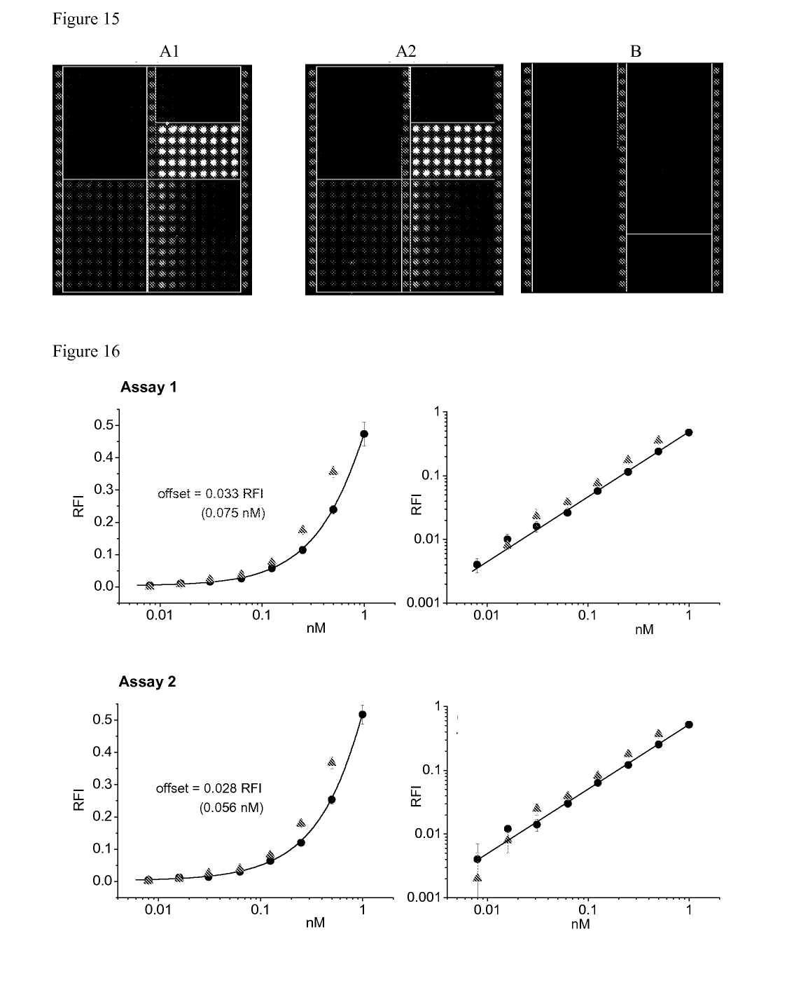

pRB assay: The assays revealed the specific signal response for dilution curves of the pRb peptide standard. Blank assay showed zero response. Signals of lysate spiked with pRb followed the signals of the standard curve in buffer at comparable, but slightly lower offset intensities (after subtraction of endogenous pRb signal level of the pure lysate). Reproducibilities of the two assays were very good. Endogenous concentration of pRb protein in pure lysate was determined by back-calculation of the blank signals of lysate from the standard curve fits. The mean concentration of endogenous protein was 0.066±0.010 nM. Other spots containing lysate 6 (negative for Histone H3) and lysate 13 (negative for pErk1/2) showed constant signals at different intensity which obviously represent their endogenous levels of pRb protein in these lysates. Good quality of fit curves achieved with correlation coefficients of r²>0.99 (FIGS. 15 and 16)

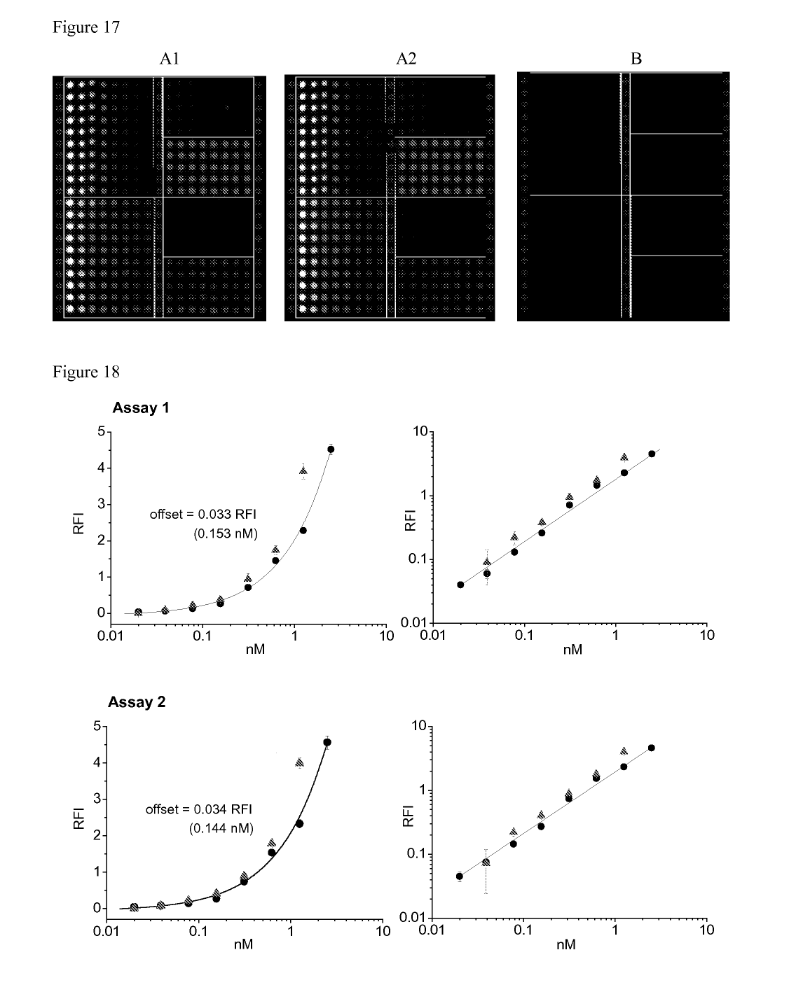

pERK1/2 assay (CST #9101): The assays revealed the specific signal response for dilution curves of the pErk1/2 peptide standard. Blank assay showed zero response. Signals of lysate spiked with pErk1/2 followed the signals of the standard curve in buffer at comparable, but slightly higher offset intensities (after subtraction of endogenous pErk signal level of the pure lysate). Reproducibilities of the two assays were very good. Endogenous concentration of pErk1/2 protein in pure lysate was determined by back-calculation of the blank signals of lysate from the standard curve fits. The mean concentration of endogenous protein was 0.149±0.005 nM. Other spots containing lysate 6 (negative for Histone H3) and lysate 12 (negative for pRb) showed constant signals at different intensity which obviously represent their endogenous levels of pErk1/2 protein in these lysates. Good quality of fit curves achieved with correlation coefficients of r²>0.99 (FIGS. 17 and 18)

pERK1/2 assay (BioSource 44-654G): The assays revealed no signal response for dilution curves of all applied peptide standards, as expected. Blank assay showed zero response. All spots containing lysates showed constant signals at different intensity which obviously represent their endogenous levels of Erk1/2 protein in these lysates.

General Remark to Effect of Increased Number of Replicate Spots:

For all 4 assays, analyzed data were compared for the effect of number of replicate spots on coefficients of variations (CVs). Mean signals of all analyte-specific signals were formed from all available number of replicate spot signals (N=5 or N=10 per condition) and from N=2 replicate spot signals (chosen form the center rows in each array field). In almost all cases, the CVs of the duplicate spot analysis were comparable or slightly smaller than for the N=5 or N=10 replicate spot analysis (to mention that this was observed at a persistently low level of CVs throughout all experiments).

### Example 6

### General Remarks

Advantages of peptide standard reagents: composition of the molecules (peptide to protein ratios) can be well prepared in a reproducible manner- concentration/number of epitope sequences per molecules could be well determined by the introduction of a small absorbance label (Dabsyl) in each peptide sequence. No adverse effects of the label were observed in the RPA assays degree of phsophorylation of synthetic peptide standards is well determined and 100% In contrary, the degree of phosphorylation of commercial recombinant protein preparations is probably largely variable and not easy to determine (see our results with several protein candidates of different vendors, for the case of Erk/pErk) peptide-protein conjugates use BSA as uniform carrier protein (BSA is a well characterized molecule for protein array applications). We expect signal responses of different epitope peptide standards not largely impacted by the (same) carrier protein properties.

In contrary, we have measured prominent differences in assay signal response from recombinant proteins of the different vendors (e.g for the case of Erk), which might be also due to the different protein preparations and characteristics (different expression systems, ±GST-tag, ±His-tag etc.)

### Competition

All 4 free forms of synthesized peptides achieved complete competition of peptide standard signals. There was a trend that phospho-peptides reached the full competition at lower concentrations which may indicative for higher affinities of the applied antibodies to phospho-epitopes. We observed no major impact or adverse effects of the competitor peptide on assay response or array quality.

In contrary, recombinant proteins used as competitors generated additional signal background on the arrays (partly by factors higher than the specific spot signals e.g. for the case of Histone H3 proteins) which made the array analysis difficult or even impossible. Nevertheless, also the recombinant proteins seemed to suppress the signals of the original standard curves. However, the recombinant proteins could not suppress the signals of the control lysates, but even generated additional signals on the lysate spots which might be due to non-specific binding to other proteins in the lysates. This implies that peptides are clearly preferable as competitor reagents.

On the lysate spots, competition with peptides could lead to complete suppression of lysate signals (e.g. for Histone H3 lysates), but also to partial (not complete) suppression leaving a basal signal even at the highest concentrations of competitor applied (e.g for pRb, pErk lysates) which might be due to a certain non-specific binding contribution of the applied antibodies. Therefore using competition and normal assays in parallel might be proposed as a universal concept to measure all future analytes-of-interest.

### Quality of Standard Curves and Assays

Standard curves generated form the dilution series printed on the RPA were generally of high quality which manifested in low CVs of replicate spots signals and good fits to data points with correlation coefficients of r²>0.99 in all cases. Standard curves of peptide reagents showed the trend for better fit correlations (smaller r² values) than standard curves of recombinant proteins.

Signal CVs of duplicate spots (N=2) and of increased number of replicate spots (N=5, N=10) were comparable indicating that standard curves from printed duplicate spots already provided optimum results.

Reproducibility of duplicate assay were also very good as manifested in low CVs of mean standard signals (array-to-array), which were in the range of a few to 10 percent. Standard curves of peptide reagents showed the trend for lower CVs (mean CV=6%) than for recombinant proteins (mean CV=9%)

Signal intensities of standard curves of the 2 total protein analytes (Histone H3 and Erk1) matched very good. Standard curves of the 2 phosphorylated protein analytes (pRb and pErk) showed lower signals for the recombinant proteins, probably due to a lower and less defined degree of phopshorylation

The figures depict the array images of the duplicate assays and the graphs of the peptide standard curves in buffer, curves of the peptide standards spiked into respective lysate and combined curves of peptide standards in buffer and spiked into lysate after correction of endogenous protein concentrations of the pure lysates.

1. A calibration reagent comprising a peptide which is attached via a linker to a protein carrier, wherein said peptide comprises an epitope of interest.

2. The calibration reagent according to claim 1, wherein the epitope of interest comprises at least one phosphorylated amino acid.

3. The calibration reagent according to claim 1 or wherein the peptide is 12 to 25 amino acids long.

4. The calibration reagent according to any one of claims 1 wherein the protein carrier is Bovine Serum Albumin (BSA).

5. The calibration reagent according to any one of claims 1, wherein the linker comprises Cysteine and 8-amino-3,6-Dioxaoctanoic acid.

6. The calibration reagent according to any one of claims 1, wherein the peptide: protein carrier ratio is between 0.3 and 1.

7. A method for generating a standard curve comprising the steps of:
a) immobilizing the calibration reagent according to any one of claims 1 in two or more concentrations on an array,
b) incubating said array with a detectable affinity reagent of interest,
c) measuring the signal intensity of the bound affinity reagent for each of the two or more concentrations of the calibration reagent, and
d) correlating the signal intensity with amount of epitope of interest.

8. A method for quantifying the concentration of a protein comprising the epitope of interest in a sample comprising
a) immobilizing on an array
i) the calibration reagent according to any one of claims 1 in two or more concentrations, and
ii) one or more biological samples
b) incubating said array with a detectable affinity reagent of interest,
c) measuring the signal intensity of the bound affinity reagent for each of the two or more concentrations of the calibration reagent and for each of the one or more biological samples,
d) correlating the signal intensity with the amount of epitope of interest, and
e) quantifying the protein comprising the epitope of interest in the one or more biological samples.

9. A method for determining the lower limit of detection of an affinity reagent of interest comprising
a) immobilizing the calibration reagent according to any one of claims 1 in two or more concentrations on an array,
b) incubating said array with a detectable affinity reagent of interest,
c) measuring the signal intensity of the bound affinity reagent for each of the two or more concentrations of the calibration reagent,
d) correlating the signal intensity with amount of epitope of interest, and
e) determining the minimum amount of the epitope of interest that can be detected with the affinity reagent.

10. A method for determining the sensitivity of the affinity reagent of interest comprising
a) immobilizing the calibration reagent according to any one of claims 1 in two or more concentrations on an array,
b) incubating said array with a detectable affinity reagent of interest,
c) measuring the signal intensity of the bound affinity reagent for each of the two or more concentrations of the calibration reagent,
d) correlating the signal intensity with the amount of epitope of interest, and thereby generating a standard curve,
e) determining the linear part of the standard curve and
f) determining the slope of the linear part of the standard curve.

11. A method for determining the dynamic range of an affinity reagent of interest comprising
a) immobilizing the calibration reagent according to any one of claims 1 in two or more concentrations on an array,
b) incubating said array with a detectable affinity reagent of interest,
c) measuring the signal intensity of the bound affinity reagent for each of the two or more concentrations of the calibration reagent,
d) correlating the signal intensity with the amount of epitope of interest, and thereby generating a standard curve,
e) determining the linear part of the standard curve and
f) determining the range of concentration of the calibration reagent of interest of the linear part of the standard curve.

12. A method for determining the specificity of an affinity reagent of interest comprising the following steps:
a) immobilizing on an array
i) the calibration reagent according to any one of claims 1 and
ii) at least one sample comprising a control peptide conjugated to protein carrier, wherein the control peptide does not comprise the epitope of interest,
b) incubating the array with a detectable affinity reagent of interest,
c) measuring the signal intensity of the bound affinity reagent on the array, and
d) comparing the signal intensity correlating with the epitope of interest of the calibration reagent with the signal intensity correlating with the control peptide.

13. The method according to claim 12, wherein the detectable affinity reagent of interest is incubated with a free epitope peptide of interest prior to step a) and wherein in step b) the array is incubated with the mixture of the affinity reagent and the free peptide.

14. The method according to any one of claims 7 to 13, wherein the calibration reagent is immobilized in the presence of matrix proteins.

15-16. (canceled)


SEQUENCE LISTING (6 sequences)

SEQ ID NO: 1 - protein, 12 aa, Homo sapiens
IQLARRIRGERA

SEQ ID NO: 2 - protein, 15 aa, Homo sapiens
  MOD_RES (6)..(6): PHOSPHORYLATION
GNIYISPLKSPYKIS

SEQ ID NO: 3 - protein, 15 aa, Homo sapiens
  MOD_RES (7)..(7): PHOSPHORYLATION
DHTGFLTEYVATRWY

SEQ ID NO: 4 - protein, 23 aa, Homo sapiens
KRITVEEALAHPYLEQYYDPTDE

SEQ ID NO: 5 - protein, 379 aa, Homo sapiens
  MOD_RES (202)..(202): PHOSPHORYLATION
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 6 - protein, 359 aa, Homo sapiens
  MOD_RES (184)..(184): PHOSPHORYLATION
AAAAAAGAGPEMVRGQVFDVGPRYTNLSYIGEGAYGMVCSAYDNVNKVRVAIKKISPFEHQTYCQRTLREIKILLRFRHENIIGINDIIRAPTIEQMKDVYIVQDLMETDLYKLLKTQHLSNDHICYFLYQILRGLKYIHSANVLHRDLKPSNLLLNTTCDLKICDFGLARVADPDHDHTGFLTEYVATRWYRAPEIMLNSKGYTKSIDIWSVGCILAEMLSNRPIFPGKHYLDQLNHILGILGSPSQEDLNCIINLKARNYLLSLPHKNKVPWNRLFPNADSKALDLLDKMLTFNPHKRIEVEQALAHPYLEQYYDPSDEPIAEAPFKFDMELDDLPKEKLKELIFEETARFQPGYRS